(19) United States
(12) Patent Application Publication
(10) Pub. No.: US 2005/0010095 A1
(43) Pub. Date: Jan. 13, 2005

(54) Multi-purpose catheter apparatus and method of use

(75) Inventors: Mark T. Stewart, Lino Lakes, MN (US); Gary R. Fiedler, Forest Lake, MN (US); Jeffrey D. Sandstrom, Forest Lake, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/837,947

(22) Filed: May 3, 2004

Related U.S. Application Data
(63) Continuation-in-part of application No. 09/848,555, filed on May 3, 2001, now Pat. No. 6,702,811.
Application No. 09/848,555 is a continuation-in-part of application No. 09/733,356, filed on Dec. 8, 2000, now abandoned.
Application No. 09/733,356 is a continuation-in-part of application No. 09/286,048, filed on Apr. 5, 1999, now Pat. No. 6,325,797.
(60) Provisional application No. 60/470,055, filed on May 13, 2003.

(51) Int. Cl. A61B 5/04; A61B 18/14

(52) U.S. Cl. 600/374; 606/41

(57) ABSTRACT

According to the present invention, a catheter having at least one multi-purpose lumen formed through the catheter terminates proximal a relatively complex-shaped distal portion thereof. In one form of this embodiment, the relatively complex-shaped distal portion comprises a looped portion having diagnostic- and/or ablation-type electrodes coupled thereto and an elongated diameter-adjusting member coupled proximal the distal end of the looped portion. The multi-purpose lumen may be used to alternately accommodate a variety of dedicated materials; such as, (i) a guide wire for initial deployment or later repositioning of the catheter, (ii) a volume or flow of a contrast media and the like, (iii) a deployable hollow needle or tube and the like used to biopsy adjacent tissue or dispense a therapeutic agent into a volume of tissue, and (iv) a cooling fluid, such as saline solution and the like dispensed at least during therapeutic tissue ablation procedures.

### CROSS-REFERENCE TO RELATED APPLICATIONS

This application claims the benefit of and incorporates by reference provisional U.S. patent application Ser. No. 60/470,055 filed 13 May 2003 and relates to related the following patent applications: U.S. patent application Ser. No. 09/848,555, filed 3 May 2001, which application is a continuation-in-part of U.S. patent application Ser. No. 09/733,356, entitled “Ablation Catheter Assembly and Method for Isolating a Pulmonary Vein,” filed on 8 Dec. 2000, which is a continuation-in-part of U.S. patent application Ser. No. 09/286,048, entitled “Ablation Catheter and Method for Isolating a Pulmonary Vein,” filed on 5 Apr. 1999 (now U.S. Pat. No. 6,325,797), each of which are hereby incorporated as if fully set forth herein.

In addition, this application hereby incorporates by reference the following co-pending non-provisional U.S. patent application; namely, Ser. No. 10/262,046 (Atty Dkt. P-10537.00) filed 2 Oct. 2002 and entitled, “Active Fluid Delivery Catheter” invented by Sigg et al.

### FIELD OF THE INVENTION

The present invention relates to a multi-purpose elongated catheter apparatus and methods of use therefor. In particular, the present invention may be used for a variety of diagnostic and therapeutic purposes as more fully described and depicted herein.

### BACKGROUND OF THE INVENTION

The heart includes a number of pathways that are responsible for propagation of signals necessary to produce continuous, synchronized contractions. Each contraction cycle naturally begins in the right atrium where a sinoatral node initiates an electrical impulse. This impulse then spreads across the right atrium to the left atrium, stimulating the atria to contract. The chain reaction continues from the atria to the ventricles after pausing briefly when passing through an atrioventricular (AV) node or junction, which acts as an electrical gateway to the ventricles. The AV node delivers the signal to the ventricles while also slowing it, so the atria can relax and desired pre-filling of the ventricles takes place prior to contraction of the ventricles.

Disturbances in the heart's electrical system may lead to various rhythmic problems that can cause the heart to beat irregularly, too fast or too slow. Irregular heart beats, or arrhythmia, are caused by physiological or pathological disturbances in the discharge of electrical impulses from the sinoatrial node, in the transmission of the signal through the heart tissue, or spontaneous, unexpected electrical signals generated within the heart. One type of arrhythmia is tachycardia, which is an abnormal rapidity of heart action. There are several different forms of atrial tachycardia, including atrial fibrillation and atrial flutter. Sometimes cardiac tissue becomes ischemic and/or a volume of essentially non-depolarizing tissue forms (i.e., a myocardial infarction or “MI”) and electromechanical response of such tissue is altered. In addition, sometimes so-called accessory pathways bridge between the ventricles and the atria causing conduction anomalies which may exacerbate a tachycardia, fibrillation, flutter or other arrhythmia condition.

With atrial fibrillation, instead of a single depolarization, numerous electrical wavefronts are generated by depolarizing tissue at one or more locations in the atria (or possibly other locations). These unexpected and typically uncoordinated electrical wavefronts produce irregular, rapid contractions of the atrial muscles and ventricles. Patients experiencing atrial fibrillation may suffer from fatigue, activity intolerance, dizziness, strokes and the like.

The precise cause of atrial fibrillation, and in particular the depolarizing tissue causing “extra” electrical signals, is currently unknown. As to the location of the depolarizing tissue, it is generally agreed that the undesired electrical impulses often originate in the left atrial region of the heart. Recent studies have expanded upon this general understanding, suggesting that nearly 90% of these “focal triggers” or electrical impulses are generated in one (or more) of the four pulmonary veins (PV) extending from the left atrium (LA). In this regard, as the heart develops from an embryonic stage, left atrium tissue may grow or extend a short distance into one or more of the PVs. It has been postulated that this tissue may spontaneously depolarize, resulting in an unexpected electrical wavefronts propagating into the left atrium and along the various electrical pathways of the heart.

A variety of different atrial fibrillation treatment techniques are available, including drugs, surgery, implants, and catheter ablation. While drugs may be the treatment of choice for some patients, drugs typically only mask the symptoms and do not cure the underlying cause. Implantable devices, on the other hand, usually correct an arrhythmia only after it occurs. Surgical and catheter-based treatments, in contrast, will actually cure the problem by ablating the abnormal tissue or accessory pathway responsible for the atrial fibrillation. The catheter-based treatments rely on the application of various destructive energy sources to the target tissue, including direct current electrical energy, radio frequency electrical energy, laser energy, and the like. The energy source, such as an ablating electrode, is normally disposed along a distal portion of a catheter.

Most ablation catheter techniques employed to treat atrial fibrillation focus upon locating the ablating electrode, or a series of ablating electrodes, along extended target sections of the left atrium wall. Because the atrium wall, and thus the targeted site(s), is relatively tortuous, the resulting catheter design includes multiple curves, bends, extensions, etc. In response to recent studies indicating that the unexpected electrical impulses are generated within a PV, efforts have been made to ablate tissue within the PV itself. Obviously, the prior catheter designs incorporating convoluted, multiple bends are not conducive to placement within a PV. Instead, a conventional “straight ended” ablation catheter has been employed. While this technique of tissue ablation directly within a PV has been performed with relatively high success, other concerns may arise.

More particularly, due to the relatively small thickness of atrial tissue formed within a PV, it is likely that ablation of this tissue may in fact cause the PV to shrink or constrict. Because PV's have a relatively small diameter, a stenosis may result. Even further, other vital bodily structures are directly adjacent each PV. These structures may be undesirably damaged when ablating within a PV.

In light of the above, an alternative technique has been suggested whereby a continuous ablation lesion pattern is formed around an inner circumference of a PV and/or within the left atrium wall about the ostium associated with the PV in question. In other words, the PV is electrically isolated from the LA by forming an ablation lesion pattern that surrounds the ostium of a pulmonary vein (herein “PVO”). As a result, any undesired electrical impulse generated within the PV could not propagate into the LA, thereby eliminating unexpected atria contraction.

Unfortunately, while PV isolation via a continuous ablation lesion pattern about the PVO appears highly viable, no acceptable ablation catheter configuration exists. Most atrial fibrillation ablation catheters have linear distal ends, designed for manipulation in a sliding fashion along the atrial wall. That is to say, the distal, electrode-carrying end of the catheter is typically slid along (or parallel to) the atrial wall. With this generally accepted configuration in mind, it may be possible to shape the distal, electrode-carrying end into a small ring sized in accordance with the PVO. For example, U.S. Pat. No. 5,617,854 discloses one such possibility. More particularly, the described ablation catheter includes a substantially ring-shaped portion sized to contact the ostium of the coronary sinus. Pursuant to conventional designs, the ring extends linearly from the catheter body. In theory, the ring-shaped portion may be placed about a PVO. However, proper positioning would be extremely difficult and time consuming. More particularly, it would be virtually impossible to locate and then align the ring about a PVO when sliding the catheter along the atrium wall. The ring must be directed toward the ostium in a radial direction (relative to a central axis of the ostium). Even if the electrophysiologist were able to direct the ring to the ostium, the periodic blood flow through the PV would likely force the ring away from the atrium wall, as the catheter body would not provide any support.

A related concern entails mapping of a PV prior to ablation. In cases of atrial fibrillation, it is necessary to identify the origination point of the undesired electrical impulses prior to ablation. Thus, it must first be determined if the electrical impulse originates within one or more PVs. Once the depolarizing tissue has been identified, necessary ablation steps can be taken. Mapping is normally accomplished by placing one or more mapping electrodes into contact with the tissue in question. In order to map tissue within a PV, therefore, a relatively straight catheter section maintaining two or more mapping electrodes must be extended axially within the PV. Ablation catheters configured to slide along the atrial wall cannot include a separate, distal extension for placement within the PV. Instead, an entirely separate mapping catheter must be provided and then removed for subsequent replacement with the ablation catheter. Obviously, these additional steps greatly increase the overall time required to complete the procedure.

Electrical isolation of a pulmonary vein via an ablation lesion pattern surrounding the pulmonary vein ostium presents a potentially revolutionary technique for treatment of atrial fibrillation. However, the unique anatomical characteristics of a pulmonary vein and left atrium render currently available ablation catheters minimally useful. Therefore, a substantial need exists for an ablation catheter designed for consistent positioning of one or more ablation electrodes about a pulmonary vein ostium, as well as for providing pulmonary vein mapping information.

In addition, when navigating to a suspected ectopic, accessory pathway, PV, PVO and the like it is often very helpful to dispense a volume of contrast media from a lumen in a catheter body so the location of the catheter can be viewed by a clinician using, for example, standard fluoroscopy techniques. Prior art techniques for dispensing contrast media, or other fluid material, either require a specialized (or additional) catheter or, if ejected from the catheter body itself, typically provide ejection of the fluid from the distal tip thereof. However, for a family of relatively complex-shaped catheters, ejecting the fluid from the distal tip may not provide optimal viewing of the distal portions thereof. Thus, a need exists for providing contrast media and the like from a location other than the distal tip for a family of relatively complex-shaped catheters.

In the field of catheter delivery of stents to a vessel of a body migration or dislodgement of the stent from an initial desired location can pose problems and risks for a patient. Accordingly, a need exists in the art of catheter delivered, and other, stents to mitigate the risks of stent migration and dislodgement.

Since many arrhythmias have a basis in a physical or tissue anomaly and given that certain therapeutic interventions are essentially irreversible, a need exists in the art to confirm or validate the status of myocytes prior to completion of certain therapeutic intervention(s). That is, a need exists in the art of cardiac therapy for providing a platform for performing electrically-guided cardiac tissue biopsies (e.g., for SA node tissue, for AV node tissue, for tissue of an Ml) in advance of or in concert with a therapeutic intervention.

In concert with or following confirmation or validation of the status of myocytes, and without moving a catheter already positioned adjacent a volume of tissue containing such myocytes the inventors hereof propose that it is beneficial to be able to provide one or more therapeutic agents (e.g., biological, pharmacological, or genetic agents) to said myocytes. Accordingly, a need exists in the art of modern day diagnostic and therapeutic electrophysiology to efficiently and accurately adjust the configuration of and parameters for diagnosing myocardial function and deliver such therapeutic agents and/or ablation therapy to a volume of previously targeted cardiac tissue.

### SUMMARY OF THE INVENTION

The present invention provides a wide variety of diagnostic and therapeutic functions, including without limitation the following: (i) diagnosing electrical pathways or providing ablation therapy for cardiac arrhythmias and/or vessels of a body; (ii) dispensing a volume of contrast media from a lumen in a catheter body proximal a distal end of the catheter so that a distal portion can be guided to a desired location using, for example, standard fluoroscopy techniques; (iii) providing a compact, adjustable-diameter loop ablation catheter for selectively creating a relatively uniform stenosis in a vessel on at least one side of a deployed stent or adjacent a location scheduled to receive a stent; (iv) providing a platform for performing electrically-guided cardiac tissue biopsies (e.g., for a myocardial infarct, an SA node, an AV node); and (v) providing electrically-guided delivery of therapeutic agents to a volume of cardiac tissue. In all forms of the present invention an elongated catheter having at least one multi-purpose lumen is readily adaptable to perform each of the forgoing procedures, among others.

One embodiment of the present invention provides a multipurpose elongated catheter assembly having a plurality of individually addressable electrodes coupled to a relatively complex-shaped, steerable distal portion. According to the present invention, the catheter has at least one multi-purpose lumen formed through a majority of the body portion of the catheter that terminates proximal the relatively complex-shaped portion thereof. In one form of this embodiment, the relatively complex-shaped distal portion comprises a looped portion having diagnostic- and/or ablation-type electrodes coupled thereto and an elongated diameter-adjusting member coupled proximal the distal end of the looped portion.

The multi-purpose lumen may be used to alternately accommodate a variety of dedicated materials; such as, (i) a guide wire for initial deployment and subsequent repositioning of the catheter, (ii) a volume of a contrast media, conductive fluids, and the like, (iii) a deployable hollow needle or tube and the like used to biopsy adjacent tissue or dispense a therapeutic agent into a volume of tissue, (iv) a cooling fluid, such as saline solution and the like dispensed at least during therapeutic tissue ablation procedures.

Another aspect of the present invention relates to the field of catheter delivered stents to a vessel of a body and a technique for presenting migration or dislodgement of a stent from an initial desired location. Accordingly, a catheter fabricated according to the present invention is used to form a vessel stenosis adjacent to at least one side of a deployed stent or adjacent a location where a stent is scheduled to be deployed.

Since many arrhythmias have a basis in a physical or tissue anomaly and given that certain therapeutic interventions are essentially irreversible, the present invention provides apparatus and techniques to confirm or validate the conductive, contractile, or functional status of a volume of myocardial tissue prior to completion of certain therapeutic intervention(s). That is, the present invention provides a platform for performing electrically-guided intracardiac tissue biopsies (e.g., for SA node tissue, for AV node tissue, for scar tissue related to an MI) in advance of or in concert with a therapeutic intervention. In this embodiment of the present invention, the catheter operates in a diagnostic mode and provides a map of intracardiac electrical activity, the presence of, and to a degree, the size (or relative center point) of, for example, an MI, an SA node, or an AV node. A deployable biopsy instrument then engages adjacent tissue and is retracted into or through the catheter body. The biopsy tissue is then immediately available for ex vivo study and may be graded, for example, according to Standardized Cardiac Biopsy Grading relative to histopathological findings on a scale of “0” to “4” as is known in the art. Alternatively, a simple gauge of “adequately perfused tissue displaying automaticity” and the like may be adequate for certain biopsy samples in the context of the present invention.

In concert with or following confirmation or validation of the status of myocytes, and without moving a catheter already positioned adjacent a volume of tissue containing such myocytes the inventors hereof believe it beneficial to be able to provide one or more therapeutic agents (e.g., biological, pharmacological, or genetic agents) to said myocytes. Accordingly, the present invention provides a platform to efficiently and accurately deliver such therapeutic agents to a volume of previously targeted cardiac tissue. Such tissue may include, for example, a portion of an MI, an SA node, or an AV node.

In summary, the present invention provides a multi-purpose elongated catheter for transvenous delivery to one or more chambers of a heart or to a location in a vessel of a body. Said catheter provides fluid delivery from a port disposed proximal the distal portion which can be used to dispense a variety of fluidic materials. These materials may be ejected directly from the port or through a dedicated, deployable hollow tube such as a relatively flexible pipette, syringe, or atraumatic (i.e., relatively blunt) needle and the like. The materials may comprise a contrast media, an solution, a biological, a pharmacological, and/or a genetic material and the like. In addition, a manually deployable and manually retractable hollow needle can have a mechanical stop so that it deploys to a predetermined depth and a valve member passively disposed near the terminus of the at least one lumen of the catheter. The multipurpose catheter according to the present invention may be deployed transvenously to diagnose electrical activity (or “map”), precisely ablate, dispense contrast media, dispense a fluidic agent proximate or injected into a volume of target tissue, and/or collect a specimen of target tissue (e.g., cardiac and/or vessel tissue) responsible for causing a variety of cardiac arrhythmias, circulatory difficulties and other related maladies.

A catheter body portion fabricated according to the present invention includes a proximal portion, an intermediate portion, and the distal end portion. The intermediate portion extends from the proximal portion and defines a longitudinal axis. The distal portion extends from the intermediate portion and includes an adjustable diameter helical portion bearing ablation- and/or diagnostic-type electrodes coupled to a remote source of ablation energy and/or electrical signal sensing equipment, respectively. According to the present invention, the active (i.e., ablation and/or diagnostic) part of the helical portion generally forms an adjustable diameter loop having a relative diameter to better accommodate a variety of different sized pulmonary vein (PV) or pulmonary vein ostium (PVO) for a given patient or patients. An optional tip portion extends distally from the helical ablation (or diagnostic) portion and is configured to help a clinician locate a pulmonary vein. The tip portion may include radio-opaque markers or other machine observable indicia so that a clinician performing a mapping and/or ablation procedure on a patient can determine the location of the catheter.

When configured for ablation, the electrodes—upon activation of the energy source—forming the ablation section disrupt existing electrical pathways by causing a suitable magnitude radio frequency signal to impinge upon adjacent tissue to form non-conducting lesions. The ablation section can form a loop portion or a distally decreasing diameter helix wherein the diameter of the helix is manually adjustable to enhance the lesion pattern for a given diameter PV. In those embodiments of the present invention having an optional tip portion, the tip provides a relatively linear leader section can have a radio-opaque ring and/or tip portion. The leader and optional tip assist a clinician when navigating the distal portion, for example near a PV or a myocardial infarct (MI) while at the same time guiding the electrodes disposed on the adjustable diameter loop or helical portion to a location disposed approximately equally from the relatively central portion of the PV or MI.

Another feature of a catheter fabricated according to the present invention relates a fluid port optionally configured with a one-way valve mechanism that reduces ingress of body fluid while also focusing the stream of fluid material emitted therefrom so it impinges upon and about the distal portion of the catheter and the adjacent tissue.

According to the present invention, catheters adapted for delivery of ablation therapy can be irrigated (e.g., via an irrigation fluid conduit for conveying fluid such as saline solution and the like from a remote reservoir) to cool the ablated tissue. In addition, catheters operated according to the present invention optionally either utilize the irrigation fluid conduit or a dedicated contrast media conduit to dispense contrast media on and about the distal portion of the catheter.

Either prior to or following deployment of a catheter according to the present invention, a clinician manipulates an elongated diameter-adjusting member (e.g., a pull wire, cable or the like) that couples to an anchoring mechanism disposed proximal the distal end of the loop portion or the decreasing diameter helix—to adjust the diameter thereof. That is, when tension is applied to the pull wire or cable the diameter of the helix decreases and when compression is applied, particularly for embodiments having a elongated resilient member (e.g., a wire) slideably constrained within an elongated substantially resilient sheath the diameter of the helical portion increases. Thus, a single catheter may be used for a variety of diameter PVO, MIs, vessels, etc. and said catheter may be advantageously manipulated to improve contact between the tissue and the distal portion of the catheter. While a single elongated member may be used to adjust the diameter of the helical portion of the distal portion, a segment of a superelastic shape memory alloy wire provides a substantially continuous diameter-restoring force tending to increase the diameter of the loop or the helical portion. The super elastic shaping wire can be advantageously mechanically coupled inside the distal portion to the outer interior diameter thereof. The pull-wire (or cable) is disposed in a lubricious conduit (and/or the same lumen as the super elastic shaping wire). To reduce the diameter one simply manually applies tension to the pull-wire (or cable). In this embodiment, a shaping wire formed of nitinol (an alloy of nickel and titanium that has the ability to return to a predetermined shape when heated).

As stated, the shaping wire can beneficially couple to the interior outer circumference of at least the majority of curvilinear portion of the helical portion. That is, the shaping wire is constrained inside an interior lumen of the helical portion on the wall portion of the lumen maximally spaced from the longitudinal axis of said loop or helical portion. Following fabrication a pull-wire of a catheter according to the present invention is subject to a slight amount of tension and heated to approximately 105 degrees Celsius for about two minutes. This process helps assure that the shaping wire and the pull-wire are situated on the proper side of the interior lumen within the distal portion (if sharing a common lumen). This process also efficiently and effectively helps the distal portion of the catheter assume and/or retain its arcuate (or curvilinear) shape.

Catheters fabricated according to the present invention are typically first deployed via a transvenous delivery catheter passed through the SVC into the RA chamber, through a relatively thin portion of tissue (e.g., the intra-atrial septum located between the RA and left atrial chamber) such as the fossa ovalis. Upon deployment of the helical portion of the catheter from the lumen of the delivery catheter, the helical portion assumes its characteristic shape and is guided toward a target location (e.g., PVO) for diagnostic mapping and/or therapeutic ablation. The diameter of the helical portion may then be adjusted (as described herein) to an appropriate diameter for the target location. Then either the tissue in contact with the helical portion may be mapped and/or ablated, as appropriate, and the helical portion of the catheter redeployed within the first target or redeployed to a second target.

### BRIEF DESCRIPTION OF THE DRAWINGS

The drawings appended hereto depict only certain illustrative embodiments of the present invention and in some, but not all of said drawings, like reference numerals are used to identify like elements. Furthermore, the drawings are not rendered to scale and thus the reader is cautioned from drawing conclusion based solely on the size or shape of the elements therein depicted. Likewise, the drawings only exemplify certain embodiments of the present invention; however, those of skill in the art will recognize variations thereof and each is intended to be conveyed hereby and covered herein as set forth in the appended claims.

FIG. 1A is an elevational view and, in part, a perspective view of a catheter assembly in accordance with the present invention.

FIG. 1B is a perspective view of a portion of the catheter assembly of FIG. 1A.

FIG. 1C is a cross-sectional view taken along lines C-C of FIG. 1A of the intermediate portion 30 of the catheter 20.

FIG. 1D is a cross-sectional view taken along lines D-D of FIG. 1A of the intermediate portion 40 of the catheter 20.

FIG. 1E depicts a partial, enlarged view of the port 37 at the terminus of the multi-purpose lumen 29.

FIGS. 2A-2B illustrates use of the catheter assembly of FIG. 1A within a heart.

FIG. 3A-3B is a perspective view of a distal portion of a catheter assembly in accordance with one form of the present invention.

FIG. 4 is a perspective view of a distal portion of a catheter assembly in accordance with one form of the present invention.

FIG. 5A depicts an alternative catheter assembly in accordance with the present invention.

FIG. 5B is a cross section of a catheter assembly of FIG. 5A along the line B-B.

FIG. 6A is a simplified, elevational cross-sectional view of a pulmonary vein and associated ostium.

FIG. 6B is a simplified, elevational cross-sectional view of an elongated diameter-adjusting member (e.g., a pull-cable or pull-wire—shown in ghost) disposed within a helical portion of a catheter and anchored proximal the distal end thereof in accordance with one form of the present invention.

FIG. 6C is a simplified, cross-sectional view of the catheter (sans the elongated diameter-adjusting member) of FIG. 6B as applied to the pulmonary vein and ostium depicted in FIG. 6A.

FIG. 7A is a simplified, elevational cross-sectional view of a pulmonary vein and associated ostium.

FIG. 7B is a simplified, elevational cross-sectional view of an elongated diameter-adjusting member (e.g., a pull-cable or pull-wire—shown in ghost) disposed within a helical portion of a catheter and anchored proximal the distal end thereof in accordance with another form of the present invention.

FIG. 7C is a simplified, cross-sectional view of the catheter (sans the elongated diameter-adjusting member) of FIG. 7B as applied to the pulmonary vein and ostium depicted in FIG. 7A.

FIG. 8 is a representational schematic of a catheter fabricated according to the present invention.

FIG. 9 is perspective view of another catheter assembly in accordance with the present invention depicting a remote fluid source and a contrast media vessel fluidly coupled to a handle and said catheter.

FIG. 10 is an enlarged elevational view of a portion of the catheter assembly depicted in FIG. 9 as viewed from the distal tip of said catheter.

FIG. 11 schematically depicts the helical portion and distal tip portion of a catheter assembly of FIG. 9 partially deployed into a pulmonary vessel (and prior to manipulation to temporarily reduce the diameter of the helical portion during said deployment).

FIG. 12 is a perspective view of a portion of an embodiment of a catheter assembly fabricated in accordance with the present invention.

FIG. 13 is a perspective view of a portion of the embodiment of a catheter assembly depicted in FIG. 12 after deployment into a pulmonary vessel (and following a first manipulation to temporarily decrease the diameter of the helical portion of the catheter and a second manipulation to at least partially reverse the temporary reduction of diameter).

FIG. 14 is a perspective view of a portion of an embodiment of a catheter assembly fabricated in accordance with the present invention.

FIG. 15 is a representational view of a vessel containing a stent and a deployable catheter according to the present invention in place to form a stenosis in said vessel adjacent said stent.

FIG. 16 is an elevational view of a deployable instrument (e.g., a biopsy collection instrument or a fluid dispensing needle) in a partially deployed location from a multi-purpose port and disposed next to a volume of tissue (e.g., a myocardial infarct) and illustrating cooperating mechanical stops so that the instrument only deploys a predetermined distance from a port.

### DESCRIPTION OF THE ILLUSTRATED EMBODIMENTS

Exemplary embodiments of the present invention shall be described with primary reference to one or more exemplary diagnostic and/or cardiac ablation catheters; however, the present invention should not be construed as so limited. Those of skill in art will readily recognize variations in the illustrated embodiments, particularly upon reflection of the Summary of the Invention set forth above. For example, in contrast to the PV mapping or ablation apparatus described herein, a similar albeit smaller scale apparatus (sans the mapping electrodes) operating at slightly less power and/or for a different amount of time can be used to ablate tissue of a vessel. Thus, tissue adjacent a stent, or a location schedule to receive a stent, forms a radial stenosis and the adjustable loop of the distal portion of the catheter is reduced and removed from said vessel. In a similar manner, a distal portion (sans any distal leader member) may be applied to diagnose electrical patterns around a surface portion of endocardial tissue (e.g., locate an MI). The diagnostic procedure may include several rounds of mapping with the loop portion adjusted to a different diameter setting. In this way the shape and electrical characteristics of the tissue may be observed. The tissue may then be precisely ablated to interrupt any potentially arrhythmic circuits formed around the MI, a biopsy may be taken, and/or a fluid material may be injected into and/or around the tissue, as desired by the clinician. In addition, the electrodes of the distal portion may be mapped by an external machine vision system such as the LocaLisa® intra-corporeal mapping system distributed by Medtronic, Inc., and/or a fluoroscopy system, and the like.

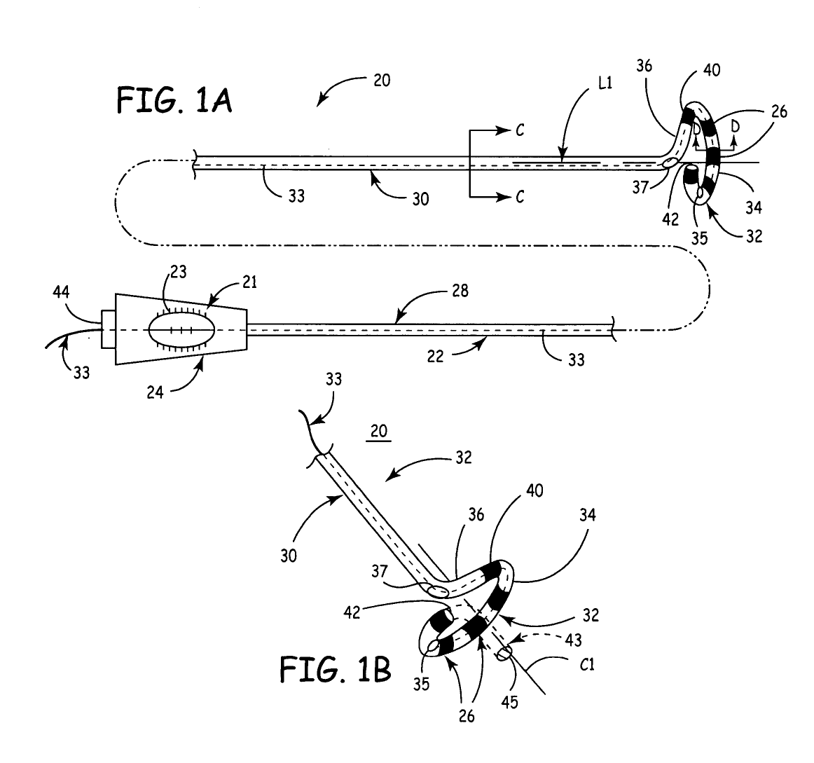

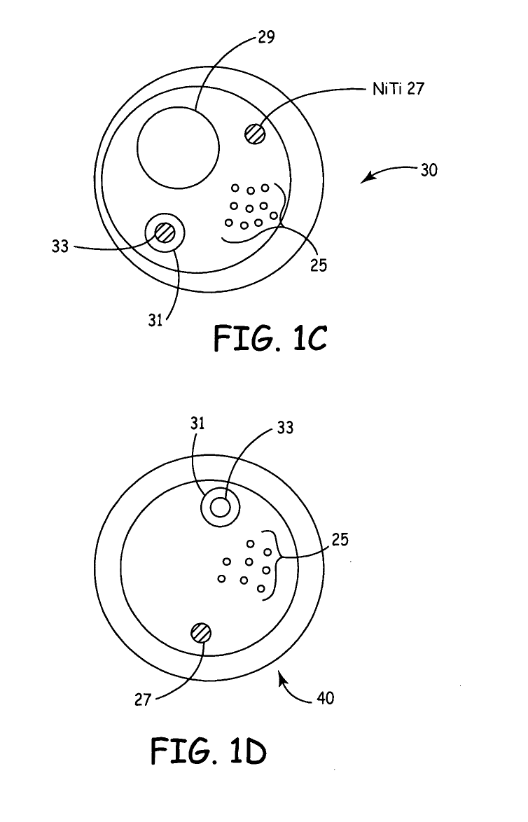

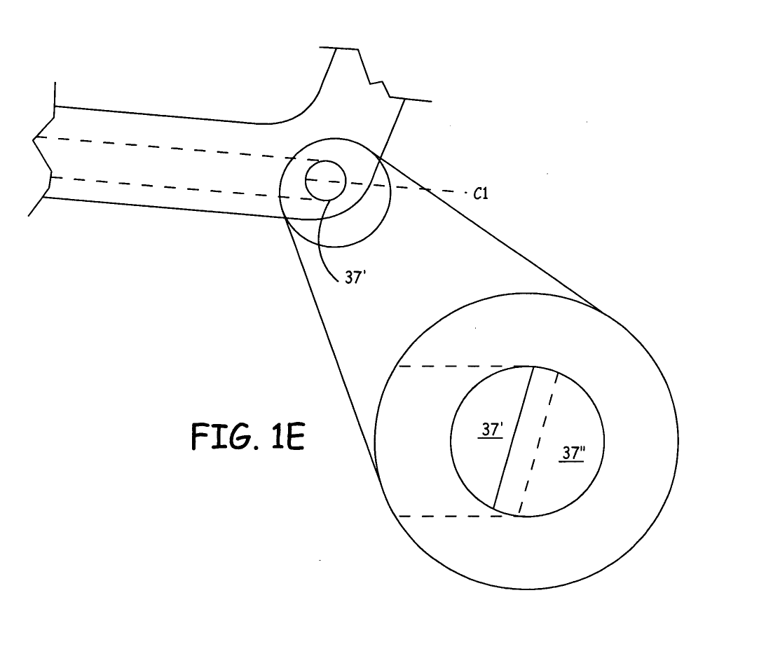

Now turning to the drawings, one embodiment of a catheter assembly 20 in accordance with the present invention is shown in FIGS. 1A-1B. The catheter assembly 20 is comprised of a catheter body 22, a handle 24 and electrodes 26. As described in greater detail below, the catheter body 22 extends from the handle 24, and the electrodes 26 are disposed along a portion of the catheter body 22. The catheter body 22 is defined by a proximal portion 28, an intermediate portion 30 and a distal portion 32, and includes a central lumen (not shown in FIGS. 1A-1B). Although not specifically shown, the central lumen of the catheter body may be configured for over-the-wire or rapid exchange applications, as well as receiving manually deployable components and fluid materials already described. In one embodiment, the proximal portion 28, the intermediate 30 and the distal portion 32 are integrally formed from a biocompatible material having requisite strength and flexibility for deployment within a heart. For intracardiac applications, the proximal portion 28 and the intermediate portion 30 each have a diameter of approximately seven French (7 F.) and the distal portion 32 has a diameter of approximately five French (5 F.). Appropriate materials are well known in the art and include various combinations of woven and polymer-based materials, such as polyamide, silicone rubber and the like. The walls of the central lumen can comprise a substantially incompressible, coiled metallic member, much longer but somewhat akin to a typical cardiac defibrillation electrode.

The intermediate portion 30 extends from the proximal portion 28. The proximal portion 28 and the intermediate portion 30 are desirably flexible, so as to facilitate desired articulation during use. In general terms, however, the intermediate portion 30 defines a longitudinal axis L1. It should be recognized that in one position (shown in FIG. 1A), the longitudinal axis L1 extends linearly through the intermediate portion 30 and the proximal portion 28. Upon deployment, it may be that the proximal portion 28 and/or the intermediate portion 30 is forced to a curved or curvilinear orientation. The distal portion 32 extends from the intermediate portion 30 and forms a looped portion 34 (depicted for convenience as a single loop). The looped portion 34 may comprise one loops or a series of decreasing diameter loops (i.e., forming a substantially circular, successively decreasing diameter helix) in a plane transverse to the longitudinal axis L1. To this end, the distal portion 32 can include a lateral segment 36. The lateral segment 36 extends in a generally lateral fashion from the intermediate portion 30. The adjustable diameter loop 34 extends from the lateral segment 36 in an arcuate fashion, turning or revolving about a central loop axis C1 (shown best in FIG. 1B). While the adjustable diameter loop 34 is shown in FIG. 1A as forming a single revolution about the central loop axis C1, the adjustable diameter loop 34 may instead include a plurality of revolutions to define a helix, a spiral or a coil. In the embodiment depicted in FIGS. 1A-1B, the central loop axis C1 is aligned with the longitudinal axis L1. Alternatively, however, the lateral segment 36 may be eliminated such that the adjustable diameter loop 34 extends directly from the intermediate portion 30. Even further, the lateral segment 36 may be configured such that the central loop axis C1 is offset from the longitudinal axis L1. Regardless of the exact construction, however, the central loop axis C1 can be disposed substantially parallel to the longitudinal axis L1. Regardless of the exact shape, the adjustable diameter loop 34 can be substantially closed and can be defined by a proximal end 40 and a distal end 42. To effectuate the “closed” configuration of the adjustable diameter loop 34, the distal end 42 can be adjacent the proximal end 40. In fact, the distal end 42 may contact the proximal end 40, although this relationship is not required. Alternatively, the distal end 42 may be longitudinally spaced from the proximal end 40. With this configuration, the distal portion 32 comprises a sufficiently flexible portion such that upon contact with a tissue wall, the distal end 42 will deflect proximally to a position adjacent the proximal end 40. In addition, as depicted in ghost in FIG. 1B, a distal leader member 43 coupled to or extending from the distal end 42 provides advantageous alignment for certain procedures (e.g., mapping or ablating a PV). In this form of the invention, a radio-opaque tip member 45 can couple to the end of the distal leader member 43. The distal tip member 43 also can align with the central loop axis C1.

As depicted in FIG. 1A and FIG. 1B, for those embodiments of the present invention having an adjustable-diameter distal or loop portion 32, an elongated diameter-adjusting member 33 (shown in ghost in FIGS. 1A and 1B, but depicted in FIG. 1C) passes through the catheter body and couples to an anchor member 35 disposed proximal the end of the arcuate portion of the loop portion 32.

Regardless of the exact shape, the adjustable diameter loop 34 can define an enclosed area A greater than a size of an ostium, other target tissue location, or vessel interior diameter (not shown) associated with a particular vessel to be isolated, as described in greater detail below. In one embodiment, the catheter assembly 20 is configured to electrically isolate a PV from the LA. With this embodiment, where the adjustable diameter loop 34 is circular, the inventors posit that only two catheters, having two ranges of adjustment, adequately cover the most common physiologic range of diameters of a PV for most all adults. That is, a distal portion having an adjustable diameter loop 34 in the range of approximately 14-22 mm or a distal portion having an adjustable diameter loop 34 in the range of approximately 18-28 mm. Of course, other sizes, either greater or smaller, are acceptable for electrical diagnosis and/or ablation of a PV or PVO. The present invention thus provides utility to clinics and clinicians by substantially reducing the inventory of and number of components required to perform a variety of procedures, as compared to the prior art.

The adjustable diameter loop 34 may be formed in a variety of ways, such as by incorporating a preformed section of super elastic, shape memory material (27 in FIG. 1C), such as Nitinol, into a loop configuration. To facilitate guiding of the distal portion 32 into a heart (not shown), the catheter assembly 20 may include a stylet (not shown) internally disposed within an optional, but desirable multi-purpose lumen (29 in FIG. 1C) of the catheter body 22. In an extended position, the stylet would extend through the distal portion 32, so as to render the adjustable diameter loop 34 straight. Upon retraction of the stylet, the distal portion 32 would form the adjustable diameter loop 34. Alternatively, the catheter assembly 20 may include a sheath (not shown) slidably receiving the catheter body 22. Prior to deployment, the distal portion 32 would be retracted within the sheath, rendering the adjustable diameter loop 34 straight. Upon deployment from the sheath, the distal portion 32 would form the adjustable diameter loop 34. Other similar approaches for providing the adjustable diameter loop 34 are similarly acceptable.

The handle 24 should be sized to be grasped by a user and includes an electrical connector 44. The electrical connector provides electrical connections to the electrodes 26 carried by the distal portion 32. To this end, a plurality of wires (25 in FIG. 1C) extend within the central lumen (see FIG. 1C) from the distal portion 32 to the handle 24. The handle 24 can have a generally longitudinally-aligned port 44 fluidly coupled to the primary lumen (29 of FIG. 1C) of the catheter 20, said port 44 adapted to: couple to contrast media dispensing apparatus, couple to an irrigation fluid reservoir, receive a guide-wire for initial deployment of the catheter, and receive a manually deployable biopsy tube, and the like. In one embodiment, a set of calibration marks 23 (hash marks or other indicia) are disposed on both a proximal portion of the member 33 and an adjacent portion 21 of the handle 24 (or other convenient location) so that a clinician can readily determine approximately the diameter of the coil 204. As depicted, a sealed window provides a means of comparing the two sets of indicia, although many other configurations may be utilized. In an additional form of this embodiment described further below, similar calibration marks are disposed on one or more of the components temporarily disposed in the multi-purpose lumen 29 (e.g., a biopsy collection instrument, a fluid dispensing hollow needle, a guide-wire, and the like) and similarly provide a clinician with information regarding the deployment of such components. Of course, as applicable these calibration indicia need to account for the depth of the (typically compressed in vivo) coil in situ. A typical depth for tissue collection during biopsy may vary but approximately 1-3 mm typically may be safely utilized. A similar dimension may be used for injection of therapeutic agents, although depending on the procedure, other dimensions may be beneficially utilized.

The electrodes 26 can comprise those types known in the art and can comprise a series of separate band electrodes spaced along the adjustable diameter loop 34. Instead of, or in addition to, separate band electrodes, the electrodes 26 may include one or more spiral or coil electrodes, or one or more counter-electrodes. Additionally, the electrodes 26 can include the following desirable characteristics: non-thrombogenic, non-coagulum or non-char forming. The electrodes 26 may be cooled by a separate source (not shown), such as a saline source. The electrodes 26 may be electrically isolated from one another, or some or all of the electrodes 26 may be electrically connected to one another. Desirably, however, at least one electrode 26 is provided. The electrodes 26 are shaped and positioned such that during an ablation procedure, a continuous, closed therapeutically-effective lesion pattern is created. Optionally, the length of each of the electrodes 26 is about 4-12 mm, but can extend about 7 mm. The spacing between each of the electrodes 26 is on the order of about 1-3 mm, with about 2 mm providing adequate function. Finally, to effectuate a continuous, closed lesion pattern, one of the electrodes 26 can be disposed at the proximal end 40 of the adjustable diameter loop 34, and another of the electrodes 26 is disposed at the distal end 42. As previously described, it is not necessary that the loop segment 38 be formed such that the proximal end 40 and the distal end 42 are integral. Instead, a slight spacing may exist. With this in mind, the spacing or gap between the electrode 26 at the proximal 40 and the electrode 26 at the distal end 42 less than about 5 mm.

FIG. 1C is a cross-sectional view taken along lines C-C of FIG. 1A of the intermediate portion 30 of the catheter 20 and depicts a representative internal configuration of some of the components of the portion 30. A super elastic shaping wire 27 is disposed inside the hollow portion 30 as are individually insulated electrode conductors 25. The conductors are shown as spaced apart members but they may be combined or braided or the like as desired. The conductors electrically couple to the electrodes 26 of the distal portion 32. A multi-purpose lumen 29 extends from the proximal portion 28 to a port 37 (depicted in FIG. 1A and FIG. 1D) and, while optional, provides a wide range of advantages as will be appreciated with reference to the drawings and text hereinbelow (particularly with reference to FIG. 1E). The elongated diameter-adjusting member 33 is depicted housed within an optional, dedicated conduit 31. The member 33 slides within conduit 31 so that tension forces and, to a degree, compression forces may be imparted to the anchor member 35 (shown in FIGS. 1A and 1B) to adjust the diameter of the loop portion 32. While the interior of portion 30 are depicted with substantial open space, an extruded or custom-shaped elongated member may be inserted into or formed during fabrication of the catheter 20.

FIG. 1D is a cross-sectional view taken along the line D-D of FIG. 1A and depicts the elongated diameter-adjusting member 33 disposed in the optional conduit 31 which are both disposed on substantially opposite sides from the shaping wire 27. As noted previously, the shaping wire can be disposed on the outer circumference of the interior of loop portion 32 while the member 33 can be disposed on the inner circumference of the interior of loop portion 32 so that the forces respectively provided thereby advantageously counteract each other and cause substantially uniform (e.g., circular) adjustment of the loop portion 32.

FIG. 1E depicts a partial view of the port 37 at the terminus of the multi-purpose lumen 29. As shown in the enlarged view of FIG. 1E, the port 37 may be configured as a passive valve member having two slightly overlapping valve flaps 37′,37″ to reduce the intrusion of body fluid to the lumen 29 and, to help focus a stream of fluid ejected from the port 37 under elevated pressure during a procedure. Such a stream of fluid may comprise a contrast media or the like which is typically dispensed into the flow of blood exiting a PV, so the added focus of the stream of fluid is especially beneficial for PV procedures. The multi-purpose lumen 29 and the port 37 aligned with the central axis of the loop portion 32 or, at least within the circumference defined by the loop portion 32. As can be appreciated, such alignment allows for a biopsy collection member, a fluid dispensing needle, and any fluid ejected from the port 37 to impinge on tissue isolated by the loop portion 32 (and in the case of fluid ejected from the port 37, also flow on and around the structure defined by the loop portion 32).

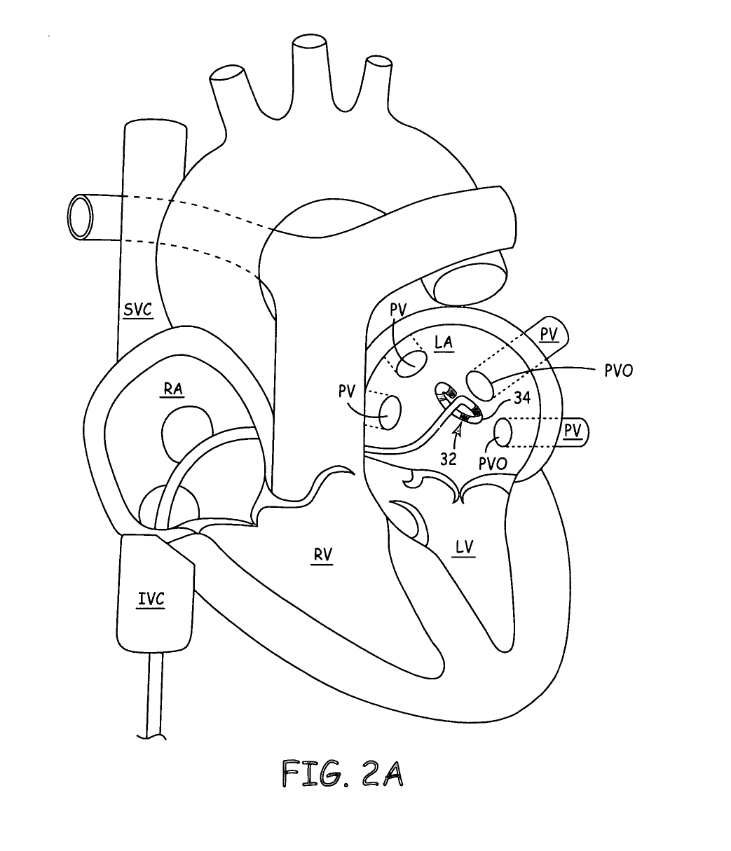

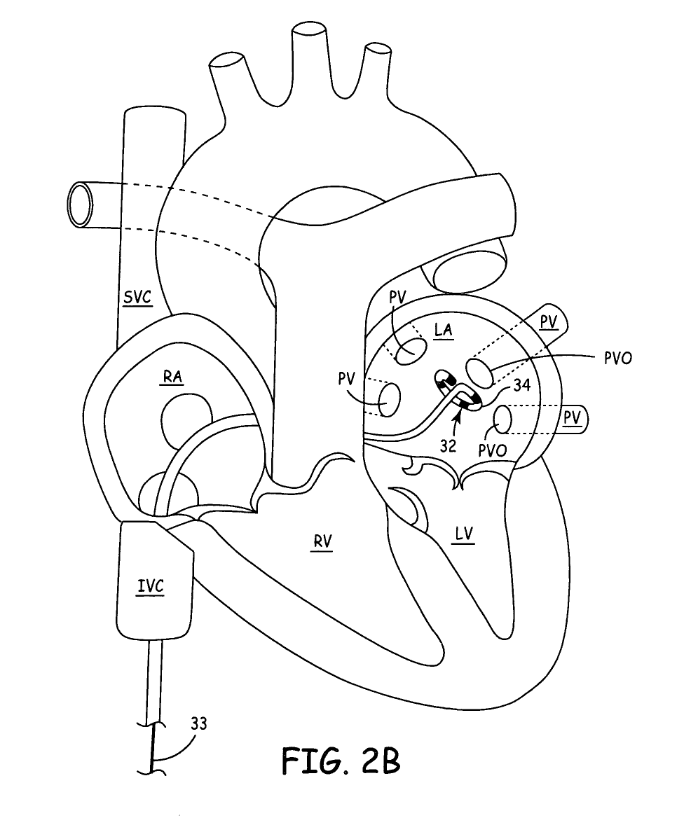

FIGS. 2A and 2B illustrate use of the catheter assembly 20 shown in FIGS. 1A-1C within a heart 50. As a point of reference, the heart 50 includes a right atrium (RA), a left atrium (LA), a right ventricle (RV) and a left ventricle (LV). An inferior vena cava (IVC) and a superior vena cava (SVC) lead into the RA. The RA is separated from the LA by an inter-arterial septum (not shown). Finally, four PVs extend from the LA. Each of the PVs forms a PVO in the LA wall. As previously described, during formation of the heart 50, it is possible that tissue of the LA may grow upwardly into one or more of the PVs. This LA tissue may spontaneously depolarize, resulting in atrial fibrillation. Notably, the heart 50 may be formed such that a separate PVO is not formed for each individual PV. In other words, a single PVO may be formed for two PVs. For example, a single PVO may be formed for both the left inferior PV and the left superior PV, with the two PVs bifurcating from the single PVO.

As shown in FIG. 2A, electrical isolation of a PV begins by directing the distal portion 32 of the catheter body 22 through the inferior vena cava IVC, into the RA through a puncture in the inter-arterial septum (not shown) and into the LA. Alternatively, the introduction of the distal portion 32 of the catheter body 22 into the RA is also suggested by passage of the distal portion 32 into the RA through the SVC. The adjustable diameter loop 34 is positioned slightly spaced from the PVO associated with the PV to be treated. More particularly, the adjustable diameter loop 34 is positioned such that the central loop axis C1 (FIG. 1B) is approximately aligned with a center of the PVO. The catheter body 22 is then advanced distally such that the adjustable diameter loop 34 contacts the LA wall about the PVO in question, as shown in FIG. 2B. In other words, the catheter body 22 is advanced in a direction parallel with the central loop axis C1 such that the adjustable diameter loop 34 contacts the LA wall, surrounding the pulmonary vein ostium (PVO). Importantly, because the central loop axis C1 is parallel to the longitudinal axis L1, the catheter body 22 longitudinally supports advancement of the adjustable diameter loop 34. In other words, the longitudinal axis L1 is effectively aligned with the PVO such that blood flow from the PV acts along the longitudinal axis L1. Thus, the catheter body 22 limits deflection of the adjustable diameter loop 34 otherwise caused by blood flow from the PV.

The electrodes 26 (shown best in FIGS. 1A-1B) are then energized to a sufficient level to ablate the contacted tissue, for example with an RF source. In one embodiment, the electrodes 26 ablate the LA tissue for 30-120 seconds at a temperature in the range of approximately 60-70 degree C. As a result, a continuous, closed lesion pattern is formed around the PVO. Pursuant to the above described catheter assembly 20 configuration, the lesion pattern is formed in a plane substantially perpendicular to the longitudinal axis L1. Notably, while the lesion pattern is shown as being only slightly larger than the PVO, the adjustable diameter loop 34 (FIG. 1A) may be sized to produce an even larger ablation lesion pattern. To this end, where a single PVO is formed for two PVs, the resulting PVO may be elongated. As shown, the adjustable diameter loop 34 (FIG. 1A) is configured to form a continuous, closed lesion pattern about the elongated-shaped PVO.

The continuous, closed lesion pattern electrically isolates the PV from the LA. Any undesired electrical impulses generated in the PV are effectively “stopped” at the lesion pattern, and will not propagate into the LA.

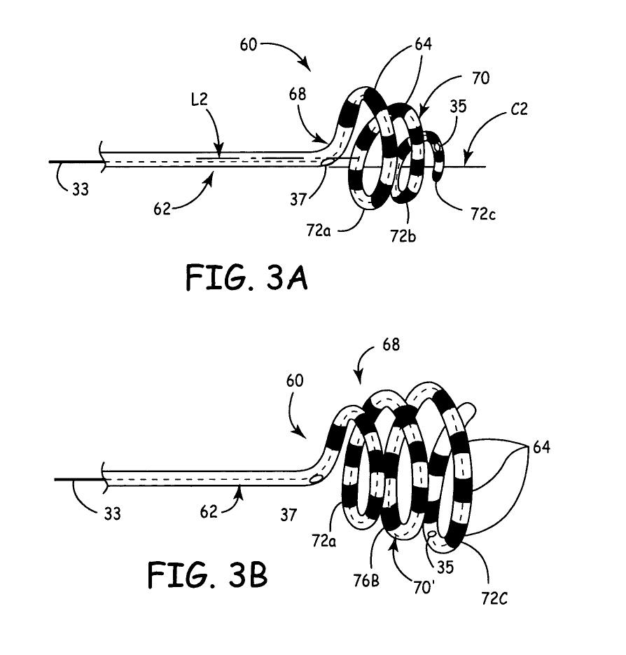

Two alternative catheter assembly structures 60 are shown in FIGS. 3A and 3B. The catheter assembly 60 includes a catheter body 62, a handle (not shown) and electrodes 64. The catheter body 62 includes a proximal portion (not shown), an intermediate portion 66 and a distal portion 68. For ease of illustration, the handle and the proximal portion of the catheter body 22 are not shown in FIGS. 3A and 3B, it being understood that these components are similar to the handle 24 and the proximal portion 28 shown in FIG. 1A. Similar to the catheter body 22, the intermediate portion 66 extends from the proximal portion and defines a longitudinal axis L2. The distal portion 68 extends from the intermediate portion 66 and forms a loop or coil 70 substantially transverse to the longitudinal axis L2 and includes a plurality of loop segments 72A-72C. The coil 70 is formed such that each of the loop segments 72A-72C revolves about a central loop axis C2. In one embodiment, the central loop axis C2 is aligned with the longitudinal axis L2 defined by the intermediate portion 66. Alternatively, the central loop axis C2 may be offset from the longitudinal axis L2. Regardless, the central loop axis C2 can be substantially parallel with the longitudinal axis L2. In these embodiments (and as depicted in ghost) the elongated diameter-adjusting member 33 passes through the catheter body 22 and is affixed to anchor member 35. The anchor member 35 is disposed, as with other embodiments herein, proximal the end of the arcuate portion (distal loop portions 72A-72C) for which an adjustable feature is desired. While not depicted in FIGS. 3A and 3B, a super elastic shaping wire can be disposed opposite the member 35 within the distal loop portions 72A-72C as previously described.

Each of the loop segments 72A-72C can define a different diameter. For example, the first loop segment 72A defines a diameter slightly larger than that of the second loop segment 72B, whereas the second loop segment 72B defines a diameter slightly greater than that of the third loop segment 72C. In this regard, while each of the loop segments 72A-72C are depicted as being longitudinally spaced (such that the loop 70 forms a multi-lane spiral or coil), the loop segments 72A-72C may instead be formed in a single plane (such that the loop 70 forms a unitary plane spiral or coil). While the loop segments 72A-72C extend distal the intermediate portion 66 so as to define a descending or decreasing diameter, an opposite configuration may also be employed. For example, FIG. 3B depicts a coil 70′ having loop segments distally increasing in diameter.

Returning to FIGS. 3A and 3B, the electrodes 64 are similar to the electrodes 26 (FIG. 1A) previously described, and can desirably comprise band electrodes disposed along the loop segments 72A-72C. In this regard, each of the loop segments 72A-72C includes electrodes 64A-64C, respectively. In one embodiment, a power source (not shown) associated with the electrodes 64 is configured to individually energize the electrodes 64 to varying levels. Further, the electrodes 64 are to be configured to provide feedback information indicative of tissue contact, such as by including a thermocouple.

The catheter assembly 60 is used in a fashion highly similar to the method previously described for the catheter assembly 20 (as shown, for example, in FIGS. 1A-1E and 2A-2C). Accordingly, a port 37 couples to a similarly situated multi-purpose lumen (not depicted) so that the catheter assembly 60 may be configured to provide all the features, configurations and advantages of the catheter assembly 20 as previously described. And, in addition, the distal portion 68 of the catheter body 62 is directed within the LA (FIG. 2A) such that the loop 70 is disposed about a PVO. It should be understood that one or more of the loop segments 72A-72C may define a diameter (or area) that is less than a diameter (or area) of the PVO in question. Conversely, the electrodes 64B associated with the second loop segment 72B (FIG. 3A) and the electrodes 64A associated with the first loop segment (FIG. 3A) are in contact with the LA wall. To avoid potential collateral damage caused by full energization of the electrodes 64C not in contact with the LA wall, each of the electrodes 64A-64C are selectively energized with a low energy supply. The energy level is not sufficient to ablate contacted tissue, but provides a low energy measurement, such as through a thermocouple or other sensing device associated with each of the electrodes 64A-64C. If the sensing device detects a temperature rise, an indication is given that the particular energized electrode 64A, 64B or 64C is in contact with tissue of the LA. Following the low energy measurement procedure, only those electrodes determined to be in contact with the LA (for example, electrodes 64A and 64B) are powered to ablate a continuous, closed lesion pattern about the PVO, as previously described.

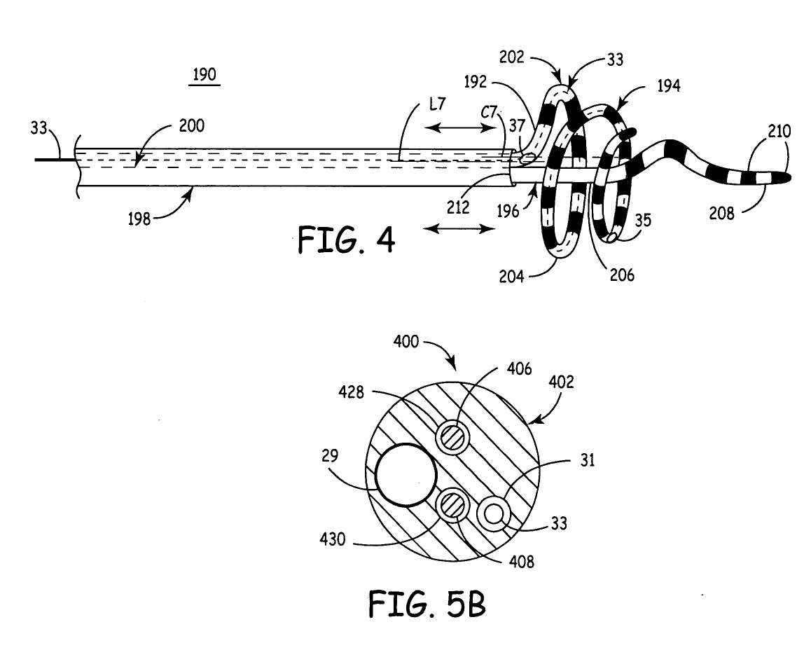

Yet another alternative embodiment of a catheter assembly 190 is shown in FIG. 4. The catheter assembly 190 includes a catheter body 192 (shown partially in FIG. 4), electrodes 194, a locating device 196, a guide catheter or sheath 198, and a port 37 at the terminus of a multi-purpose lumen (not depicted). As described in greater detail below, the sheath 198 retains the catheter body 192 and the locating device 196 such that each of the catheter body 192 and the locating device 196 are slidable between a retracted position and a deployed position (shown in FIG. 4).

The catheter body 192 is virtually identical to the catheter body 62 (FIG. 3A) and catheter 20 (FIG. 1A-1E) as previously described and includes a proximal portion (not shown), an intermediate portion 200 defining a longitudinal axis L7 and a distal portion 202. The distal portion 202 extends from the intermediate portion 200 and forms an adjustable diameter coil or plurality of adjustable diameter loops 204 substantially transverse to the longitudinal axis L7. Alternatively, the adjustable diameter coil 204 may form a single adjustable diameter loop. In any case, a port 37 forms the terminus of a multi-purpose lumen (not specifically depicted) thereby providing all the advantages of said lumen and port 37 (and optional valve 37′,37″) previously described and depicted herein. The coil 204 revolves around a central loop axis C7, that, in one embodiment, is aligned with the longitudinal axis L7. Disposed at least through the distal portion 202, and in particular the coil 204, an elongated segment of preformed super elastic material (not shown) couples to an outside circumference portion. Also, an elongated diameter adjusting member 33 is slideably disposed from beyond the proximal portion, throughout the intermediate portion 200 and mechanically couples to anchor member 35 proximal an end portion of one of said loops or coil 204. As with previous embodiments the member 33 can reside on an opposing inner surface of the coil 204. Thus, the super elastic material provides a biasing force tending to increase the diameter of the one of said loop or coil 204 and when tension is applied to the elongated member 33 overcoming the biasing force, the diameter of said loop or coil 204 decreases. The adjustable diameter loop portion can be sufficiently flexible so as to assume a relatively straight configuration when retracted within the sheath 198.

Further, during fabrication of the adjustable diameter loop or coil 204 of the distal portion 202 the super elastic material, the elongated member 33 and related components are heat treated at an elevated temperature with tension applied to the member 33. Thus, the loop or coil 204 readily and efficiently assumes a predetermined, smaller-diameter shape. Thereafter the memory characteristics of the super elastic material tends to expand the diameter until it is placed into a sheath 198 for later therapeutic deployment. Of course, when deployed from the sheath 198, the distal portion 202 generally assumes the shape of a coil 204 as shown in FIG. 4.

While the diameter of the coil 204 may be adjusted at any time following deployment from sheath 198, and tension is applied to member 33 just prior to advancing the coil 204 into contact with a pulmonary vein or ostium thereof. The tension is decreased after the coil 204 enters a PV and, in this embodiment, the diameter of the coil 204 automatically increases due to the biasing force provided by the super elastic material.

In one form of this embodiment, the member 33 comprises a resilient pull-wire and the super elastic material is only optionally present. Thus, after applying tension to the member 33 and advancing the coil 204 to a desired position, a compression force is applied to the member 33 thereby increasing the diameter a desired amount. In a related embodiment, and as described previously, a set of calibration marks (hash marks or other indicia) are disposed on both a proximal portion of the member 33 and an adjacent portion of the handle 24 (or other convenient location) so that a clinician can readily determine approximately the diameter of the coil 204. In an additional form of this embodiment, similar calibration marks are disposed on one or more of the components temporarily disposed in the multi-purpose lumen 29 (e.g., a biopsy collection instrument, a fluid dispensing hollow needle, and the like) and similarly provide a clinician with information regarding the deployment of such components. Of course, these calibration indicia need to account for the depth of the (typically compressed in vivo) coil in situ. A typical depth for tissue collection during biopsy may vary but approximately 1-3 mm typically may be safely utilized. A similar dimension may be used for injection of therapeutic agents, although depending on the procedure, other dimensions may be beneficially utilized.

In another form of this embodiment, the member 33 comprises a resilient elongated member slideably disposed within a lubricious sheath. Thus, a multi-stranded wire cable disposed in a coiled sheath may be used to transmit tension and/or compression forces to the anchor 35 to thereby adjust the diameter of the coil 204. Again, the super elastic material is optionally present, but not required. Thus, after applying tension to the cable 33 and advancing the coil 204 to a desired position, a compression force is applied to the cable 33 thereby increasing the diameter a desired amount. Alternative materials may of course comprise the elongated member 33 in this and other embodiments; accordingly, elongated composite materials, metallic materials, extruded materials, resin-based materials and combinations thereof having sufficient resiliency may be used. Also, while not required member 33 or the multi-purpose lumen (29) may be used to transmit electrical energy and/or signals to and from the electrodes coupled to the coil 204.

Turning again to FIG. 4, electrodes 194 are identical to those previously described and can comprise band electrodes disposed along the coil 204. Alternatively, a continuous coil electrode or counter-electrode may be provided.

The locating device 196 is relatively rigid and includes a shaft 206 defining a tip 208 that supports the mapping electrodes 210. The shaft 206 is sized to be slidably received within a lumen (not shown) in the sheath 198. As shown in FIG. 4, the tip 208 can assume a coil shape with decreasing diameter and while specifically depicted in conjunction in illustration of locating device 196 of FIG. 4 said device 196 may of course be adapted to house the elongated member 33 and anchor 35 so that the decreasing diameter is adjustable too. Alternatively, the tip 208 may be substantially straight. However, the tip 208 can be sufficiently flexible such that upon retraction into the sheath 198, the tip 208 assumes a relatively straight form. Additionally, the tip 208 has a shape memory or super elastic characteristic such that upon deployment from the sheath 198, the tip 208 assumes the coiled shape shown in FIG. 4. For example, the tip 208 may include stainless steel or Nitinol core wires. Further, the tip 208 may be formed from a shape memory alloy of Nitinol that forms the coil shape when heated above a certain temperature. The heat may be achieved through resistive heating of the wire directly, or by surrounding the wire with a tubular heater.

The sheath 198 includes a proximal end (not shown) and a distal end 212, and forms at least one central lumen (not shown) sized to maintain the catheter body 192 and the locating device 196. Alternatively, a separate lumen may be provided for each of the catheter body 192 and the locating device 196. Regardless, the sheath 198 is configured to slidably maintain each of the catheter body 192 and the locating device 196 in a relatively close relationship. In one embodiment, the sheath 198 is formed of a relatively soft material such as 35D or 40D Pebex.

As described above, each of the catheter body 192 and the locating device 196 are slidable relative to the sheath 198. In a deployed position (depicted in FIG. 4), the distal portion 202 of the catheter body 192 and the tip 208 of the locating device 196 extend distally from the sheath 198. More particularly, the locating device 196 is positioned such that the tip 208 is distal the coil 204. In this extended position, the tip 208 is essentially aligned with the central loop axis L7.

During use, the catheter body 192 and the locating device 196 are retracted within the sheath 198. The sheath 198 is then guided to the LA (FIG. 2A-2B). The catheter body 192 and the locating device 196 are deployed from the sheath 198. More particularly, the distal portion 202 of the catheter body 192 and the tip 208 of the locating device 196 are extended from the distal end 212 of the sheath 198 (as shown in FIG. 4). A locking device (not shown) is provided to secure the catheter assembly 190 in the deployed position. As previously described, upon deployment, the distal portion 202 forms the coil 204, whereas the tip 208 can assume a coil shape. The tip 208 locates and is directed axially into a PV as previously described. The mapping electrodes 210 sample electrical activity of the pulmonary vein tissue. If the mapping procedure determines that the PV requires electrical isolation, the sheath 198 is guided in a direction along the central loop axis C7 until the coil 204 contacts the LA (FIG. 2A-2B) wall about the PVO (FIG. 2A-2B). Because the catheter body 192 and the locating device 196 are directly connected by the sheath 198, the tip 208 effectively positively centers the loop 204 about the PVO. The electrodes 194 may be selectively energized with a low energy supply to determine which of the electrodes 194 are in contact with tissue of the LA. Some or all of the electrodes 194 are then energized to ablate a continuous, closed lesion pattern about the PVO, thereby electrically isolating the PV from the LA.

While the catheter assembly 190 has been described as including the sheath 198 to maintain the catheter body 192 and the locating device 196, the sheath 198 may be eliminated for example, the catheter body 192 may alternatively be configured to include lumen (not shown) sized to slidably receive the locating device 192. In this regard, the locating device 192 may serve as a guide wire, with the catheter body 192 riding over the locating device 192 much like an over-the-wire catheter configuration commonly known in the art. Even further, the catheter body 192 may include a rapid exchange design characteristic for quick mounting to removal from the locating device 190.

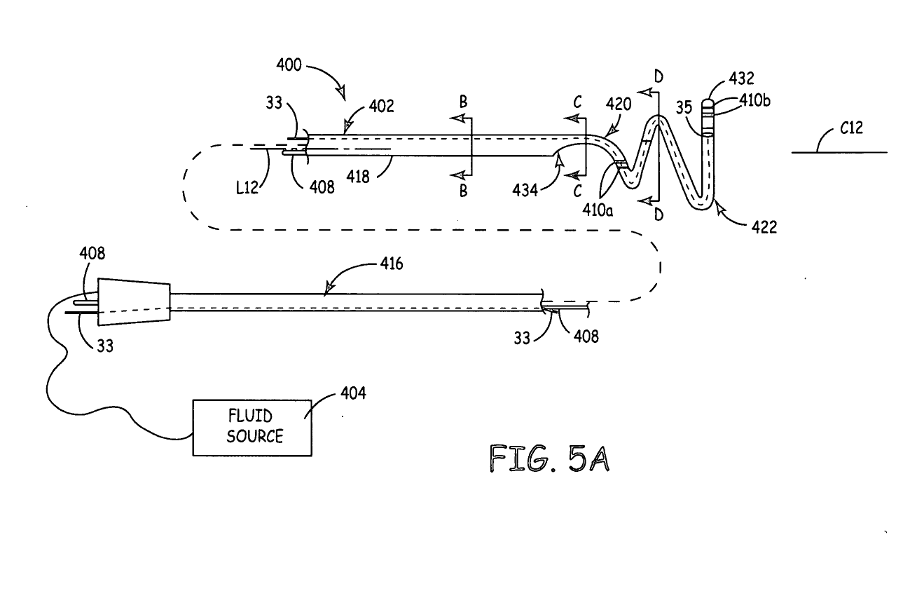

Yet another alternative embodiment of a catheter assembly 400 is shown in FIG. 5A. With reference to FIG. 5A, the catheter assembly 400 includes a catheter body 402, a fluid source 404, a shaping wire 406 (shown in FIG. 5B) coupled proximal to the distal portion 420, a combined coil-deflection guide wire 408, an elongated diameter-adjusting member 33 (shown primarily in ghost in FIG. 5A) coupled to an anchor 35, and first and second sensing electrode pairs 410a and 410b. The fluid source 404 fluidly connects to one or more irrigation lumens (not shown) formed in the catheter body 402.

The distal end of deflection guide wire 408 and of the elongated diameter-adjusting member 33 are each mechanically fastened to different locations of the catheter 400 (e.g., anchor 35 and location 434, respectively) so that when manipulated they cause the configuration of the distal end 420 to change. With respect to member 33 the location of anchor 35 is very important; that is, the anchor 35 can be proximal (i.e., spaced from) the distal tip portion 432. Thus, manipulation of the guide wire 408 tends to deflect the entire distal end 420 while manipulation of member 33 adjusts the diameter of the loop or helical portion 422. Accordingly, each of the combined coil-deflection guide wire 408 and the elongated diameter-adjusting member 33 are slidable between a retracted position and a deployed position. That is, each has a retracted and deployed position and when manipulated therebetween the wire 408 causes deflection of a distal portion 420 (and components distal said portion 420) and the member 33 causes a change in the relative diameter of an ablation section 422 of said distal portion 420. Finally, the sensing electrodes 410a,410b are secured to a portion of the catheter body 402.

The fluid source 404 is shown schematically in FIG. 5A, and can assume a wide variety of forms. The fluid source 404 maintains an appropriate volume of a conductive liquid or ionic fluid, such as a hypertonic saline solution, and includes a pump (not shown). The pump is controllable to provide a desired flow rate of the liquid to the catheter body 402.

The catheter body 402 includes a proximal portion 416, an intermediate portion 418, and a distal portion 420. Construction of the catheter body 402 is described in greater detail below. In general terms, and as shown in FIG. 5A, the distal portion 420 extends from the intermediate portion 418 and forms, or is formed to, an adjustable-diameter coil or helix (e.g., conical or cylindrical). Further, the distal portion 420 defines an ablation section 422. The ablation section 422 forms, or is formed to, a loop of at least one revolution. As with previous embodiments, the adjustable diameter loop formed at or by the ablation section 422 revolves around a central loop axis C12, that is substantially parallel to and desirably aligned with a longitudinal axis L12 defined by the intermediate portion 418. Alternatively stated, the adjustable diameter loop formed at or by the ablation section 422 extends transversely relative to the longitudinal axis L12. The port 434 forms the terminus of multi-purpose lumen 29 (depicted in FIG. 5B) and serves the purposes previously described for this embodiment of the present invention.

With additional reference to FIG. 5B, which is a cross-sectional view taken along the line B-B of FIG. 5A, the catheter body 402 can comprise at least a first lumen 428 and a second lumen 430. The first lumen 428 extends from the proximal portion 416 through the distal portion 420, including the ablation section 422, and can be closed (or terminate) at a distal end 432 of the catheter body 402. As described in greater detail below, the first lumen 428 is sized to relatively tightly contain super elastic shaping wire 406 as depicted in FIG. 5B, and can have a diameter only slightly greater than that of the shaping wire 406 and any other elements carried by the shaping wire 406, such as a coil electrode. Within the helical ablation section 422, the shaping wire 406 mechanically couples to the outer circumference therein (and is generally opposed to and spaced from the elongated diameter-adjusting member 33). With this configuration, the first lumen 428 provides sufficient spacing about the shaping wire 406 to allow passage of the conductive liquid or ionic fluid (not shown) from the fluid source 404. Thus, the first lumen 428 is fluidly connected to the fluid source 404 and directs liquid from the fluid source 404 to at least the ablation section 422. By closing the first lumen 428 at the distal end 432, a back pressure can be generated within the first lumen 428 to promote fluid irrigation through the ablation section 422 as described below. As mentioned in conjunction with the description of FIG. 5A, the multi-purpose lumen 29 is depicted disposed in an arbitrary location of the catheter body 402 and serves the same functions as previously mentioned with respect to other embodiments depicted herein. Finally, the member 33 is depicted as slidingly disposed within optional lumen 31 and spaced from shaping wire 406, which is not as critical for the section of the catheter body 402 as it is for the distal portion 422 (depicted in FIG. 5A) for the reasons previously stated.

The second lumen 430 extends from the proximal portion 416 to the distal portion 420 and terminates proximal the distal end 420. The second lumen 430 is sized to slidably secure the guide wire 408. The guide wire 408 mechanically couples in the vicinity of location 434 so that when manipulated predictable deflection of the distal portion 420 occurs. As is known in the art, in lieu of a single guide wire 408, two or more guide wires may be configured to couple to opposing locations of the intermediate portion 418 enabling three-dimensional deflectable motion to the distal portion 420.

The catheter body 402 will now be described in greater detail with reference to fluid irrigation. For ease of illustration, only a portion of the catheter body 402 is provided in FIG. 5A, including the intermediate portion 418 and the distal portion 420. Further, the distal portion 420 is shown in a straightened or uncoiled state, as compared to the helical configuration of FIG. 5A. As previously described, the catheter body 402 includes the ablation section 422 formed along the distal portion 420. In one embodiment, the ablation section 422 is formed of a material different from a remainder of the catheter body 402, including the distal portion 420. More particularly, the ablation section 422 is tubular, formed of a flexible, microporous, surgically-safe material, whereas a remainder of the catheter body 402, and in particular the distal portion 420, is formed of a flexible, fluid impermeable material. In one embodiment, the ablation section 422 is a microporous polymer, one desirable material is micro-porous, high density, expanded polytetrafluoroethylene (PTFE), whereas a remainder of the distal portion 420 is a fluid impermeable polymer, such as polyethylene, polyurethane or PEBAX™ material (polyurethane and nylon). A remainder of the catheter body 402 is similarly formed from a fluid impermeable, polymeric, electrically non-conductive material but can be more rigid than the distal portion 420. Alternatively, other known materials useful in catheter applications are equally acceptable.

Use of a porous material for the ablation section 422 establishes a plurality of pores 440 extending from an interior surface 442 to an exterior surface 444. As shown in FIG. 5D, the pores 440 are in fluid communication with the first lumen 428. It should be noted that a size of the pores 440 has been greatly exaggerated in FIG. 5D for purposes of illustration. Further, the pores 440 need not be continuous from the exterior surface 444 to the interior surface 442. Instead, a plurality of interconnected interstitial spaces can be formed by the ablation section 422 so as to establish fluid communication between the interior surface 442 and the exterior surface 444. As a point of reference, a porosity of the ablation section 422 is in the range of approximately 5-25 microns. Regardless of the exact construction, the ablation section 422 formed with microporous material irrigates liquid (and contained ions) from the first lumen 428 to the exterior surface 444 in a uniform fashion along an entirety of the exterior surface 444, or at least along an entire length of the ablation section 422 (and thus into contact with targeted tissue (not shown)). With this construction, then, where the conductive fluid has been energized (such as by an electrode), a continuous electrode is effectively established along an entire length of the ablation section 422, in direct contrast to “compartmentalized” ablation electrodes typically employed. By way of example, use of a high density, expanded PTFE material for the ablation section 422 having a straightened length of approximately 3.2 inches (81.3 mm) and wall thickness of approximately 0.010 inch (0.25 mm) exhibited virtually uniform liquid distribution of a hypertonic saline solution along the exterior surface 444 at flow rates as low as 1 ml/min.

While the ablation section 422 has been described as being formed of a microporous polymer, other constructions and/or equivalents thereof are equally acceptable. For example, an alternative ablation section 450 is initially formed as a non-porous sleeve. During manufacture, a series of small passages 452 are created in the sleeve, such as with a laser, to facilitate generally uniform irrigation of a conductive liquid for an interior to an exterior of the sleeve. Once again, the passages 452 are minute, having a diameter in the range of 5-100 microns. A wide variety of materials are useful for the sleeve, including polyethylene (high or low density), nylon, polyamide block co-polymer, PTFE, polyurethane, fluoropolymers, etc. Regardless of exact construction, in a embodiment the distal portion 420, including the ablation section 422, comprises a compliant portion, and can readily be manipulated to a desired shape. To this end, the shaping wire 406 is employed to selectively direct the distal portion 420 to the helical or coiled configuration of FIG. 5A. Thus, in one embodiment, the distal portion 420, including the ablation section 422, defines the first lumen 428 for receiving the shaping wire 406 along with an electrode (not shown) for applying an ablation energy to fluid irrigated through the ablation section 422. This relationship is depicted in FIG. 5D. Alternatively, and with reference to FIG. 5E, an additional lumen, such as a third lumen 460, can be formed in the distal portion 420 (and extending to the proximal portion 418). With this configuration, the first lumen 428 is available to direct fluid to the ablation section 422, while the third lumen 460 is available to maintain the shaping wire 406 and/or an electrode for applying an ablation energy. Even further, the material selected for the distal portion 420 can have an elasticity or shape memory characteristic such that the helix configuration is independently achieved by the distal portion 420 without requiring the separate shaping wire 406. Regardless of the exact construction, the ablation section 422 is sized so as to provide a relatively large ablation area when formed as a loop (as otherwise depicted in FIG. 5A). In one embodiment, the ablation section 422 has a straightened length in the range of approximately 2-8 inches (51-203 mm), with a value of approximately 5 inches (127 mm) being acceptable. Alternatively, other dimensions are equally acceptable.

The shaping wire 406, and in particular the distal segment thereof, can be formed of a thin material having a super elasticity or shape memory characteristic. For example, in one embodiment, the shaping wire is formed from spring-like material such as super elastic or pseudo-elastic nickel titanium (commercially available as Nitinol material), having a diameter in the range of approximately 0.010-0.020 inch (0.25-0.5 mm). With this or other resilient material (such as stainless steel or resilient plastic), the desired helical configuration of the distal segment is imparted during formation of the shaping wire. As a result, the distal segment has a highly resilient, spring-like attribute whereby the distal segment can be “forced” to a substantially straight state, but will readily revert to a loop or helical configuration of the various embodiments herein.

Optionally, a metal wire may be wound about a portion of the shaping wire to form a coil electrode and may be secured to the shaping wire, such as by a weld or a mechanical fixture or clamp and the like. Further, such a metal wire couples to a power source such as a source of radio frequency (RF) energy. The location and length of such a coil electrode relative to the shaping wire corresponds with a location and length of the ablation section relative to the catheter body 402. Thus, upon final assembly and activation of the power source, the coil electrode serves to provide an ablation energy to the ablation section, and in particular, a conductive fluid otherwise supplied to the ablation section. Notably, a winding density and thickness of the coil electrode does not impede the ability of the distal segment of the shaping wire to revert to the loop-shaped or helical configurations elsewhere described herein. In the straightened state, the coil electrode has a length dimension slightly greater than a length of the ablation section, in the range of approximately 2.5-8.5 inches (63-216 mm). In one embodiment, with the ablation section length of approximately 5 inches (127 mm), the coil electrode 474 has a length of approximately 5.5 inches (140 mm). A wide variety of known, electrically conductive materials are available for use as the metal wire 470. However, the metal wire 470 can comprise other materials (e.g., platinum, copper, copper-silver alloy, nickel-cobalt alloy, etc.).

While the shaping wire has been described as carrying a single metal wire, and thus a single coil electrode, multiple wires/coil electrodes can be provided. For example, in another embodiment, six metal wires forming six coil electrodes are each secured to the distal segment as previously described. The coil electrodes are longitudinally spaced by approximately 1-2 mm. The coil electrodes can be sized such that when the shaping wire assumes the helical shape, each of the coil electrodes have a length less a full revolution defined by the distal segment. While the coil electrodes may have varying lengths, the coil electrodes are typically sized such that a combined length is slightly greater than one revolution (or of a length of the ablation section). With this configuration, a user can selectively ablate quadrants or portions of a complete circle (or other closed shape) by selectively energizing less than all of the coil electrodes. For example, a user may wish to ablate only muscle tissue (determined by electrogram analysis). By providing multiple, relatively short coil electrodes, this desired procedure is available. Once again, however, only a single coil electrode is necessary.

Returning to FIG. 5A, the guide wire 408 is of a type known in the art, and can be constructed of a rigid metal material. Further, the guide wire 408 is sized to be slidably received within the second lumen 430 of the catheter body 402 but anchored, for example, in the vicinity of location 434. With this relationship, the guide wire 408 selectively articulates the distal portion 420. As described below with reference to an alternative embodiment, the guide wire 408 is not central to the present invention but adds a convenient mode of deflecting the distal portion toward a desired location, particularly in vivo.

Finally, the sensing electrode pairs 410a, 410b comprise band electrodes capable of providing feed back information indicative of electrical activity. As described below, the sensing electrode pairs 410a, 410b are useful for evaluating the “completeness” of an ablation pattern formed by the catheter assembly 400. To this end, the sensing electrode pairs 410a, 410b are strategically located along the distal portion 420 relative to the ablation section 422. It will be noted that the distal portion 420 can be helically-shaped, having a decreased diameter proximal the ablation section 422, and an increased diameter distal the ablation section 422. With this in mind, the first sensing electrode pair 410a can be located proximal the ablation section 422 for evaluating electrical activity “within” the loop pattern defined by the ablation section 422. Conversely, the second sensing electrode pair 410b is distal the ablation section 422 for evaluating electrical activity “outside” the loop. With alternative embodiments, one or both of the sensing electrode pairs 410a, 410b can be eliminated; or additional sensing electrodes provided. Even further, additional sensors, such as a thermocouple, can be included along the distal portion 420. As with other embodiments, the axis of the multi-purpose lumen 29 can align with the central loop axis so that any components and any fluid materials deployed from the port 434 are disposed within the outer periphery defined by the loop portion 422.

For embodiments of the present invention adapted for tissue ablation, a fluid source couples to the ablation section and provides fluid flow at a rate of approximately 4-10 ml/min. After waiting for a short period to ensure increased fluid flow to, and irrigation through, the ablation section, the operative electrode or electrodes are energized, for example with RF energy. This energy is transferred via the fluid irrigated along the ablation section to the tissue contacted by the ablation section. The conductive fluid establishes a conductive path from the coil electrode to the contacted tissue, thereby ablating the targeted tissue. As previously described, a porosity associated with the ablation section is such that the conductive fluid irrigates or “weeps” or “sweats” to the exterior surface of the ablation section. This weeping attribute serves to cool the coil electrode and, because the fluid contacts the targeted tissue, minimizes the opportunity for thrombosis formation. In one embodiment, the coil electrode is energized for two minutes at 40-50 watts, although other ablation energies and times are equally acceptable. The endpoint of energy delivery can be determined by the reduction in electrogram amplitude at the discretion of the physician.

Prior to and following application of the ablation energy, the catheter assembly 400 is typically operated in a diagnostic mode to determine the electrical characteristics of the target tissue and, later, whether a closed, electrically isolating ablation pattern has been established around the target tissue (e.g., in the chamber wall, about or outside of an ostium, around an MI, etc.). More particularly, a pair of sensing electrode are simultaneously interrogated to evaluated isolation of the PV from the LA wall. Conversely, an ablation pattern formed on a tissue wall, as well as locations of the sensing electrode pairs relative to the ablation pattern when the distal portion is compressed against the tissue wall. With these orientations in mind, a first of the sensing electrode pair is located on one side of the ablation pattern, whereas a second sensing electrode of the pair is located on the other side of the ablation pattern. This configuration is further exemplified in which the first sensing electrode pair is located within loop defined by the ablation section, whereas the second sensing electrode pair is outside of the loop. Following application of the ablation energy, the sensing electrode pairs are operated to observe and sense electrical activity inside and outside of the ablation pattern. If it is determined that electrical activity continues to traverse the ablation pattern, an ablation energy can again be applied to the coil electrode to further ablate the tissue wall about the PVO. Once sufficient ablation has been achieved, the catheter body and the guide wire are retracted from the PVO. Subsequently, additional ablation patterns can be formed about other ones or all of the PVOs.

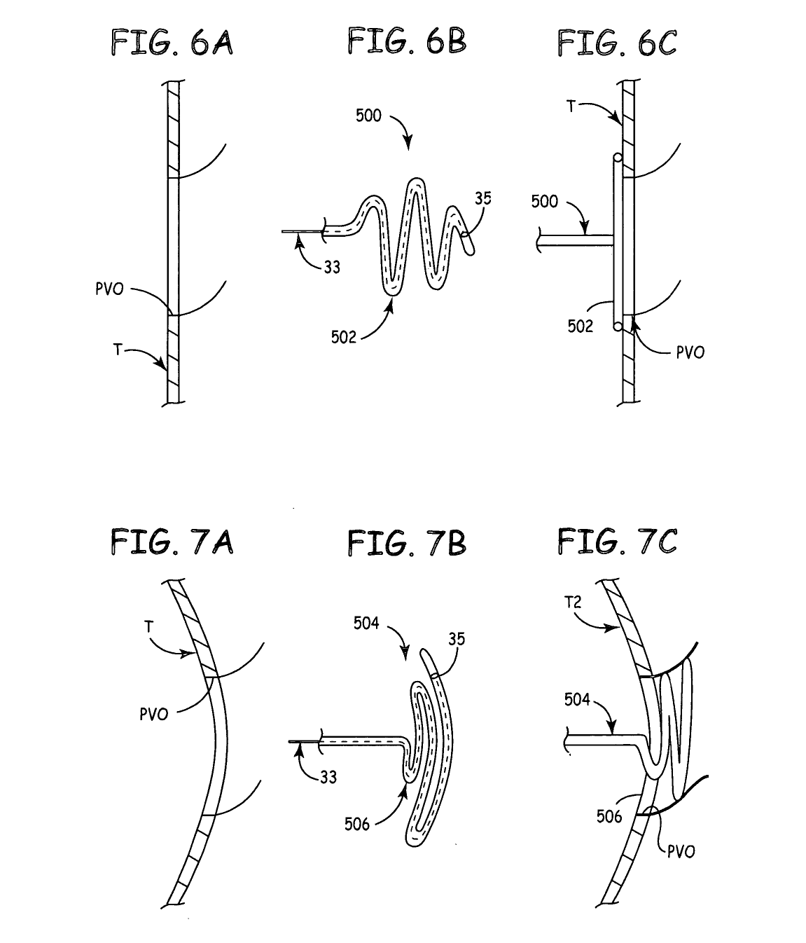

Alternatively, as shown in FIGS. 6A and 7A, a PVO and associated chamber wall tissue (T) often have a non-planar shape. More particularly, pulmonary vein ostia are often formed to have a “saddle” shape. When so identified, a user will select a correspondingly-shaped shaping wire, such as a shaping wire (not shown) so that the helical portion 504 depicted in FIGS. 6B and 7B promotes sufficient contact between the deployed helical portion 504 and the interior tissue of the PV. The shaping wire includes a coiled segment 502,506 respectively that, when axially compressed and/or when tension is applied to elongated diameter-adjustment member 33, assumes a compact, helical shape having a reduced diameter. During use, and when axially compressed against a PV, the coiled segment 506 assumes a reduced diameter “saddle” shape corresponding generally to the interior tissue of the PV and/or of (or surrounding) the PVO, as depicted in FIGS. 6C and 7C. In practice, by providing a single catheter capable of assuming a variety of shapes and sizes (e.g., loop or helical diameter dimensions) a user can quickly ablate and electrically isolate all of the PVs without removing the catheter assembly 500,504 from the LA. This compares very favorably to prior art approaches wherein a number of interchangeable, but uniquely sized and shaped shaping wires were inserted and retracted in a time-consuming effort to perform the same task.

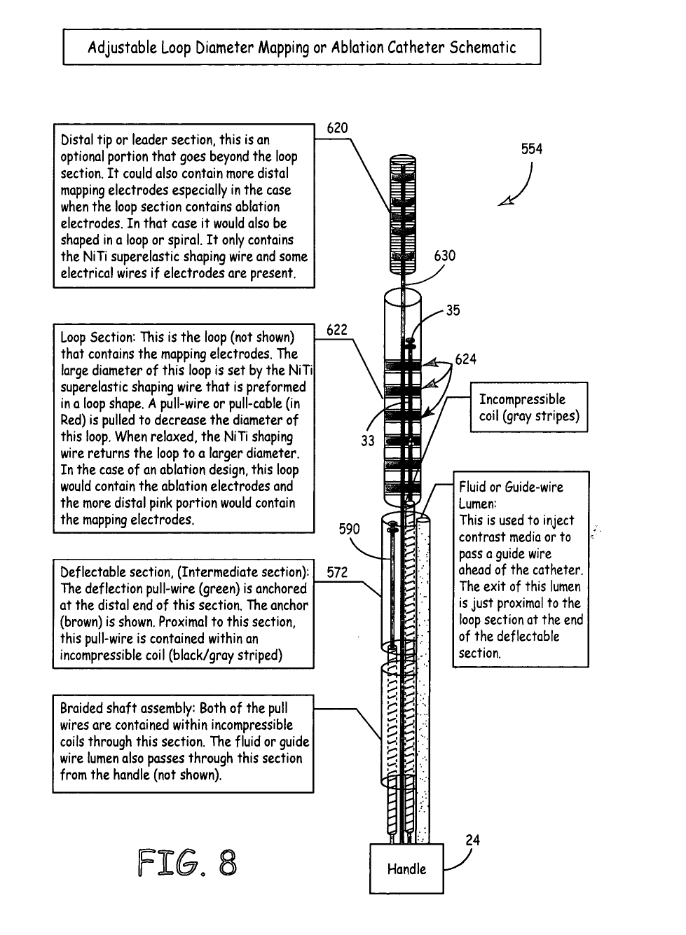

Now turning to the schematic representation of FIG. 8, it can be appreciated that the mapping or ablation catheter 554 includes a distal tip portion 620 that extends distal from an ablation section 622, desirably configured into at least one loop member. In one embodiment, the ablation catheter 554 is highly similar to the catheter body 402 (FIGS. 5A-5B) previously described, such that the ablation section 622 is formed from a microporous material that is fluidly connected to a fluid source (not shown) by a lumen (not shown). Further a shaping wire 630 (as depicted extending the full longitudinal length of the catheter 554) similar to that previously described is fixedly disposed within the ablation catheter 554 for selectively forming (i.e., biasing toward a greater diameter for) the loop portion 622, and in particular the arcuate ablation-electrode bearing loop portions of section 622, to the helical or loop configuration, and electrode(s) 624 associated with the ablation section 622. Alternatively, the ablation catheter 554 can be formed in accordance with any other of the embodiments disclosed herein.

With the ablation catheter 554 deployed, the distal tip portion 620 is then maneuvered to locate the orifice in question, for example one of the pulmonary vein ostia. In this regard, a user can steer the delivery catheter 554 both proximally and distally. For example, the first pull wire 590 can be manipulated or tensioned to bend the catheter 554 at the intermediate region 572. This first bend serves to “aim” or direct the distal tip portion 620 generally toward the orifice (or ostium) of interest and it is then maneuvered or directed toward the ostium, the distal tip portion 620 itself can steered via tensioning of a second pull wire (now shown) so as to facilitate exact, desired positioning of the distal tip portion 620 within the ostium. Once the distal tip portion 620 has been positioned within the ostium in question, the ablation catheter 554 is advanced, with the distal tip portion 620 effectively “guiding” the ablation section portion 622, to the target site. Once positioned, the ablation catheter 554 is available to map and/or form a continuous ablation pattern on the chamber-wall outside of/around the pulmonary vein ostium as previously described. If a PVO requires electrical isolation, the distal tip portion 620 can be readily aligned with the desired ostium by steering or bending of the delivery catheter 554 both proximal and distal the opening 560 as previously described.

A few illustrative examples of interior components of catheters fabricated according to the present invention are now described. The intermediate portion (FIG. 5A) can be formed of a material different from that of the proximal portion. More particularly, unlike the reinforced, torqueable composition of the proximal portion, the intermediate portion can comprise a softer material such as nylon, polyurethane or “PEBAX”™. With this configuration, the intermediate portion is highly amenable to bending via tensioning of a first pull wire. A length of the intermediate portion and the location of a mechanical anchor for the first pull wire dictates the focal point at which the intermediate portion deflects when tension is imparted to the pull wire, as well as an available bend radius. In an embodiment, the intermediate portion has a longitudinal length in the range of 5-25 cm, more desirably about 15 cm.

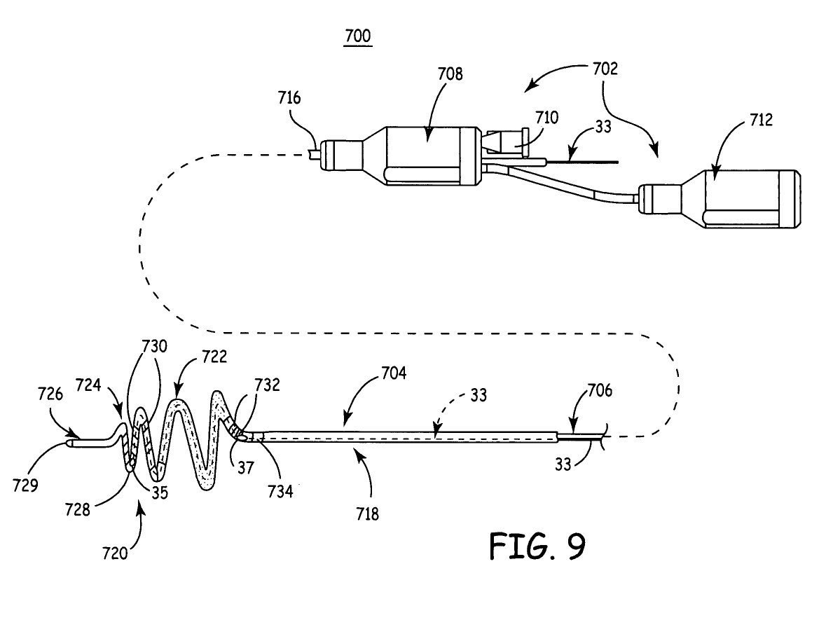

Yet another alternative embodiment of a catheter assembly 700 is shown in FIG. 9. In an embodiment, the catheter assembly 700 includes input components 702, a catheter body 704, an elongated diameter-adjusting member 33 mechanically coupled to an anchor 35 and a shaping wire 706 (shown partially in FIG. 9). In addition, a port 37 forms the terminus of a multi-purpose lumen 29 (not depicted) as with other embodiment of the present invention hereof. In general terms, the input components 702 are connected to the catheter body 704, and control functioning of the catheter assembly 700. As with several previous embodiments, the shaping wire 706 can be fixed within a lumen (not shown) of the catheter body 704 during fabrication when the elongated member 33 is under tension so that the distal end of the catheter body assumes a desired loop or helical shape.

The input components can assume a wide variety of forms relating to desired functioning of the catheter assembly 700. For example, in one embodiment, the input components 702 include a hand piece 708, a fluid input port 710 and an ablative energy source 712 (only a portion of which is depicted in FIG. 9). As previously described, the catheter assembly 700 can be configured to ablate tissue by energizing fluid irrigated from a portion of the catheter body 704. The hand piece 708 provides fluid flow to the catheter body 704 via the fluid input port 710. For example, a saline or other fluid source can be connected to the fluid input port 710. Similarly, the ablative energy source 712 includes an electrical connector (shown in FIG. 9) electrically connecting an energy source (not shown) to corresponding components of the catheter assembly 700 (such as internally disposed coil electrode(s) not otherwise illustrated) via the hand piece 708. In this regard, electrical connectors are well known in the art.

Alternatively, and as described below, where the catheter assembly 700 is designed to make use of a differing ablation energy technique, one or both of the fluid input port 710 and/or the electrical connector 712 can be eliminated, modified or replaced with an appropriate component. For example, the catheter assembly 700 can be configured to ablate tissue via energized band or coil electrodes, ultrasound, RF energy, microwave energy, laser, cryogenic energy, thermal energy, etc., as is known in the art.

The catheter body 704 includes a proximal portion 716, an intermediate portion 718 and a distal portion 720. As with previous embodiments, the intermediate portion 718 extends from the proximal portion 716 and defines a longitudinal axis. The distal portion 720, in turn, extends from the intermediate portion 718, and includes an ablation section 722 and a tip 724. The tip 724 extends distally from the ablation section 722, and, in one embodiment, terminates in a leader section 726.

The shape of the distal portion 720 is an important feature of the catheter body 704. In particular, at least a segment of the distal portion 720 defines a distally decreasing- and adjustable-diameter helix. In this regard, the ablation section 722 generally forms at least one loop that can be disposed transverse to the longitudinal axis defined by the intermediate portion 718. With the embodiment of FIG. 9, the ablation section 722 forms a plurality of loops that define the distally decreasing- and adjustable-diameter radius helix. This configuration has surprisingly been found to greatly enhance positioning and ablation about a PVO (not shown). The ablation section 722 can define a plurality of loops curving approximately 5400. Alternatively, any other degree of circumferential extent is acceptable, ranging from 900-7200. It has surprisingly been found that curving the ablation section approximately 5400 ensures a complete, closed lesion pattern with minimal power requirements. Further, the frontal diameter defined by the ablation section 722 is sized to initially be larger than a pulmonary vein ostium. For example, in one embodiment, a maximum outer diameter defined by the ablation section 722 is approximately 35 mm. Thus, as the ablation section 722 is poised for deployment to a PV, the diameter is temporarily reduced by applying tension to the elongated member 33. Then, the ablation section 722 is advanced to a desired location within the PV and the tension is released or at least reduced. In this way the ablation section 722 establishes adequate mechanical contact with the adjacent PV tissue thereby promotion efficient and effective ablation lesion patterns. Alternatively, other maximum outer diameters corresponding with pulmonary vein ostiums are acceptable. However, the maximum frontal outer diameter defined by the ablation section 722 is in the range of 10 mm-35 mm.

The tip 724 includes a proximal section 728 that continues the distally decreasing- and adjustable-diameter helix otherwise defined by the ablation section 722. That is to say, a relatively uniform decreasing radius helix is defined by the ablation section 722 and the proximal section 728 of the tip 724. However, the proximal section 728 of the tip 724 can be rendered not capable of ablating tissue during an ablative procedure at the ablation section 722, as described below. The proximal section 728 of FIG. 9 defines a maximum frontal outer diameter approximating a diameter of a pulmonary vein, +/−10 mm. With this configuration, the proximal section 728 is sized for placement within a pulmonary vein (not shown).

Finally, the leader section 726 extends distally from the proximal section 728 and can be relatively linear. To this end, the leader section 726 can be coaxially aligned with, or angled with respect to, a central axis defined by the intermediate portion 718. Stated otherwise, the relatively linear leader section 726 can be angled with respect to, alternatively aligned with, a central axis defined by the helix of the ablation section 722 proximal section 728. By employing a relatively linear or straight design, the leader section 726 more readily locates a pulmonary vein, and is easily maneuvered within a pulmonary vein. Further, the relatively linear design is easily identified on an appropriate viewing device, such as a fluoroscope, such that the leader section 726 serves as an indicator of venous branching.

In addition to the varying shapes defined by the ablation section 722 and the tip 724, other differing features are provided. For example, in another embodiment, the catheter body 704 is highly similar to the catheter body 402 (FIGS. 5A-5B) previously described, such that the ablation section 722 is formed from a microporous material, such as expanded PTFE, that is fluidly connected to the fluid input port 710 by a lumen (not shown). Further, the shaping wire 706, similar to that previously described, is slidably disposed within the catheter body 704 for selectively forming the distal portion 720 to the shape illustrated in FIG. 9. In one embodiment, and as previously described, the shaping wire 706 positions a coil electrode(s) at the ablation section 722. Alternatively, the coil electrode(s) can be independently maintained within the ablation section 722 apart from the shaping wire 706. Regardless, in this configuration, the ablation section 722 is porous, whereas the tip 724 is impermeable to fluid flow. The tip 724, and in particular the leader section 726, can be formed of a soft, atrumatic material such as low durometer polyurethane. Thus, the tip 724, and in particular the leader section 726, has a lower durometer than a remainder of the catheter body 704, and will not cause trauma to contacted tissue (e.g., pulmonary vein). To further soften the leader section 726, the distal-most section of the shaping wire 706 (otherwise disposed within and “shaping” the leader section 726) can be taper ground to a smaller diameter than a remainder of the wire 706.

An additional feature of the catheter assembly 700 is the inclusion of an electrode 729 on the leader section 726; spaced electrodes 730 (referenced generally in FIG. 9) along the proximal section 728 (i.e., distal the ablation section 722 and proximal the leader section 726); electrodes 732 adjacent, but proximal, the ablation section 722; and an electrode 734 along the intermediate portion 718. In one embodiment, each of the electrodes 729-734 is a band electrode capable of sensing electrical activity, as known in the art. As such, each of the electrodes 729-734 is electrically connected to a device (not shown) otherwise associated with the catheter assembly 700 for analyzing signals generated by the electrodes 729-734, and can comprise an ECG reference electrode. Thus, the electrodes 729-734 serve as reference electrodes, available for confirming complete ablation as described below. Alternatively, or in addition, one or more of the electrodes 729-734, and in particular the electrodes 730, serve as pacing electrodes. Even further, one or more of the electrodes 729-734 can be formed from a radio opaque material (e.g., platinum-iridium) or other material viewable using available devices, such as a fluoroscope.

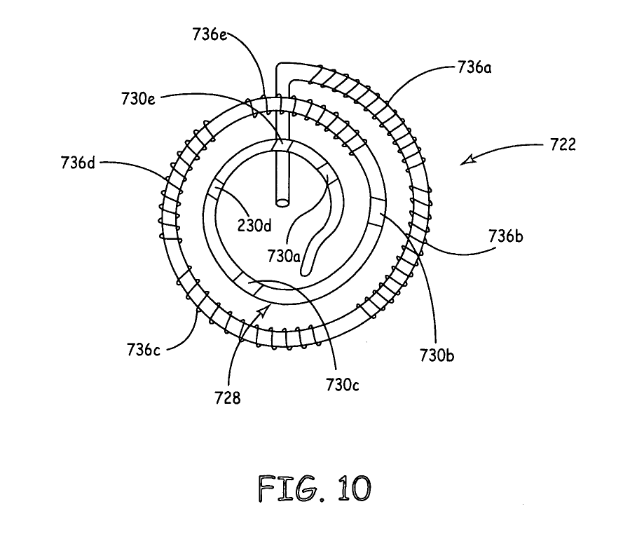

In another embodiment, the electrodes 730 along the proximal section 728 of the tip 724 are located at specific radial locations of the formed helix. The location of each of the electrodes 730 correlates with a radial location of respective ones of the coil electrodes (not shown) relative to the helix of the ablation section 722. This relationship is best illustrated by the diagrammatic view of FIG. 10 in which a frontal representation of the decreasing radius helix otherwise defined by the ablation section 722 and proximal section 728 is provided. FIG. 10 includes, by way of example, five coil electrodes 736a-e disposed along the ablation section 722, and five of the reference electrodes 730a-e disposed along the proximal section 728. The coil electrodes 736a-e can comprise a material such as platinum-iridium, although a wide variety of other conductive materials are equally acceptable. Each of the reference electrodes 730a-e are radially aligned with a respective one of the coil electrodes 736a-e. Of course, any other number of coil electrodes 736 and reference electrodes 730 is equally acceptable, and more than one reference electrode 730 can be provided along the helix of the proximal section 728 and correlated with one of the coil electrodes 736. Regardless, as described in greater detail below, the spatially spaced and correlated nature of the coil electrodes 736 and the reference electrodes 730 facilitates selective ablation of specific portions of tissue (i.e., extra-ostial), as opposed to complete, “closed” ablation pattern.

Returning to FIG. 9, as is clear from the above, though the tip 724 extends directly from the ablation section 722, several differences exist. More particularly, and in one embodiment, the ablation section 722 and the tip 724 have a number of differing features, including shape, material, porosity, and durometer. Alternatively, the catheter body 704 can be configured such that the ablation section 722 and the tip 724 differ only in terms of shape, material, porosity, or durometer. Thus, for example, the catheter body 704 need not be configured to form the ablation section 722 with a microporous material. Instead, any of the other configurations previously disclosed herein can be incorporated. Along these same lines, the ablation section 722 can be configured to accommodate a variety of different ablative energy sources other than energize irrigated fluid. In one embodiment, the ablation section 722 delivers an RF energy, and is a single electrical element or multiple elements each defining a portion of the circumference of the ablation section 722, each in the range of 10°-540°, each in the range of 45°-180°.

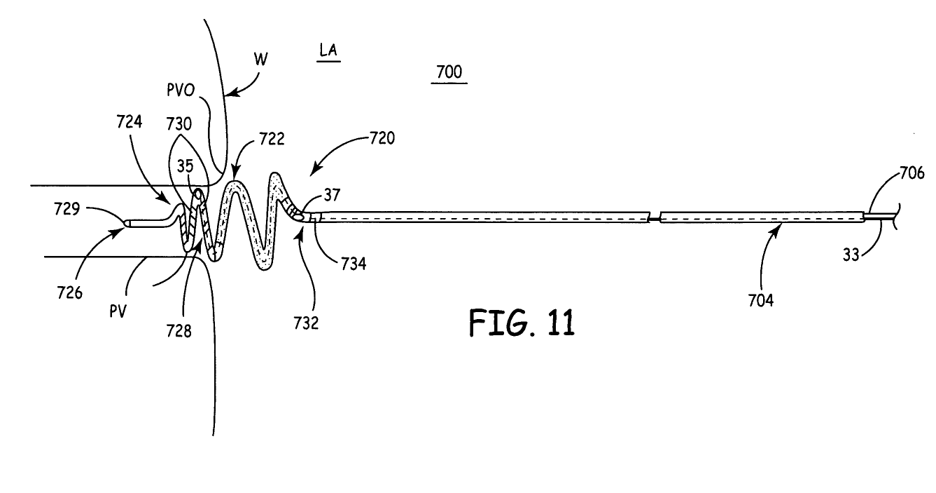

With reference to FIG. 11, the distal portion 720 is, following previously described preparatory and deployment steps, positioned within the LA. As a point of reference, FIG. 11 generally illustrates a portion of the LA and includes an atrium wall (W) and a PV. The PV forms a PVO at the wall W. With this general description in mind, the tip portion 724 is employed to locate the PV. The relatively linear leader section 726 is easily positioned within the PV. Once the pulmonary vein has been located, the diameter of the distal portion 720 is temporarily reduced by application of tension to elongated diameter-adjustment member 33 and then distal portion 720 is advanced into the PV. Then the tension is released or at least reduced until the ablation section 722 contacts the tissue wall W in or about the PV (and PVO). In this regard, due primarily to the location of the anchor 35 the tip portion 724 readily slides along and within the PV and the diameter of only the coiled portion of the distal portion proximal portion 724 is adjusted. The tip portion 724 can be formed of an biologically inert material such that contact between the tip portion 724 and the PV does not damage the PV tissue. Further, the distally decreasing- and adjustable-diameter helix formed by the proximal section 728 of the tip 724 contacts the PV wall, effectively seating the distal portion 720 within the PV. Once seated, the ablation section 722 is essentially centered about the PVO. Subsequently, as the ablation section 722 is optionally manually compressed against the wall W (not depicted), a more complete ablation perimeter is consistently defined about (proximal) the PVO (or extra-ostial).

Once properly positioned, extra-ostial ablation via the ablation section 722 is initiated. For example, with one embodiment and as previously described, an appropriate fluid is irrigated through the ablation section 722, and is then energized via the coil electrode(s) (not shown), for example with RF energy. This energy is transferred, via the fluid irrigated along the ablation section 722, to the tissue contacted by the ablation section 722. The conductive fluid establishes a conductive path from the coil electrode(s) to the contacted tissue, thereby enhancing the effects of the ablation energy on the targeted tissue. Depending upon operator preference and indications of electrical activity recorded from the electrodes 730, it is possible to selectively ablate only specific portions of the extra-ostial perimeter by applying energy only to specific ones of the coil electrodes. In some instances, the atrial tissue fibers extend into the PV along only a portion of the PVO circumference. The operator may desire to only ablate at this specific location, as opposed to forming a complete, closed ablation pattern. The catheter assembly 700 of the present invention promotes this procedure. In particular, and with additional reference to FIG. 10, the various reference electrodes 730a-e can be interrogated to determined where electrical activity is occurring relative to a circumference of the PVO. The corresponding coil electrode(s) 736a-e are then energized to effectuate partial, or quadrant ablation.

Following application of the ablation energy, the catheter assembly 700 can be operated to determine whether a closed, electrically isolating ablation pattern has been established in the chamber wall W, about or outside of the PVO. More particularly, one or more of the electrodes 729-734 are interrogated to evaluate electrical isolation of the PV from the atrium wall W. The electrodes 729 along the tip 724 provide information relating electrical activity within the PV, whereas the electrodes 732, 734 provide information relating to electrical activity within the LA. Thus, where the electrodes 729-734 are ECG reference electrodes, a comparison can be made between the electrical activity within the PV (via the electrodes 730) and the electrical activity with the LA (via the electrodes 732, 734) or electrical activity sensed from a catheter placed in the coronary sinus. If it is determined that electrical activity within the PV is similar or otherwise related to electrical activity at the LA, further ablation of the tissue wall W is required. Ablation energy can again be applied to further ablate the tissue wall W about the PVO.

Once sufficient ablation has been achieved, the diameter of one or more of the loops of the helical portion of distal section 720 is reduced via application of tension to member 33 and then is retracted from the PV. Because the reduction in diameter occurs nearly perpendicular to the adjacent surface of the PV tissue, the adjustable diameter feature promotes relatively safe and efficacious means of removal of an ablation catheter. That is, compared to prior art ablation catheter removal technique (e.g., applying tension to a part of the catheter so that the fully deployed ablation section pulls away substantially parallel from the tissue surface), the present invention provides a means of first reducing contact between the ablation section and the tissue prior to extracting the catheter from a PV.

Another advantage to the present invention relates to the fact that a single catheter can be used for a patient having diverse sized and shaped PV and related ostia. That is, additional PV diagnostic and ablation therapy can be performed on other ones or all of the pulmonary vein ostia PVOs of such a patient. In addition, the present invention allows physicians to use one or at most a few catheters fabricated according to the present invention (e.g., each having a adjustable range of diameters suitable for children, young adults and large adults).

As previously described, the catheter assembly 700 can assume a wide variety of shapes and helix diameters beyond the specific embodiments depicted herein and can include a port 37 forming the terminus of multi-purpose lumen 29 so that the assembly 700 provides all the benefits previously described herein. For example, the catheter assembly 700 can be configured to provide the distally decreasing- and adjustable-diameter helical shape of the ablation section 722 and the tip 724 via a component other than the shaping wire 706. Alternatively and/or in addition, a delivery catheter or sheath can be provided. Even further, the catheter assembly 700 can be provided with one or more pull wires as previously described to effect directional deflection.

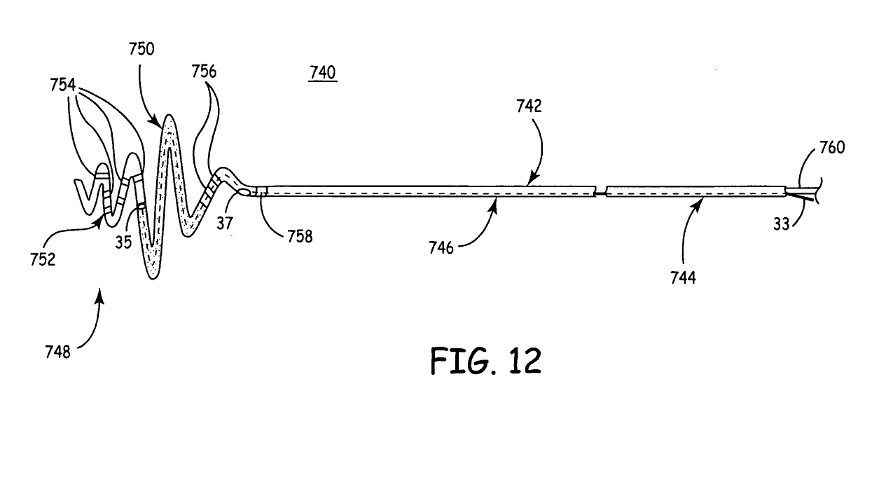

Yet another alternative embodiment catheter assembly 740 is shown in FIG. 12. For ease of illustration, only a distal region of the catheter assembly 740 is depicted. The catheter assembly 740 is similar to the catheter assembly 700 (FIG. 9) previously described, and includes a catheter body 742. The catheter body 742 includes a proximal portion 744, an intermediate portion 746 and a distal portion 748. The proximal portion 744 is connected to an ablative energy source (not shown), such as that previously described. The intermediate portion 746 extends from the proximal portion 744 and defines a longitudinal axis. Finally, the distal portion 748 extends from the intermediate portion 746 and forms an ablation section 750 and a tip portion 752. An elongated diameter-adjusting member 33 is slideably disposed within the proximal, intermediate and at least a majority of the ablation section 744,746,750 and mechanically coupled to an anchor 35 that is fixed at a location proximal the tip portion 752.

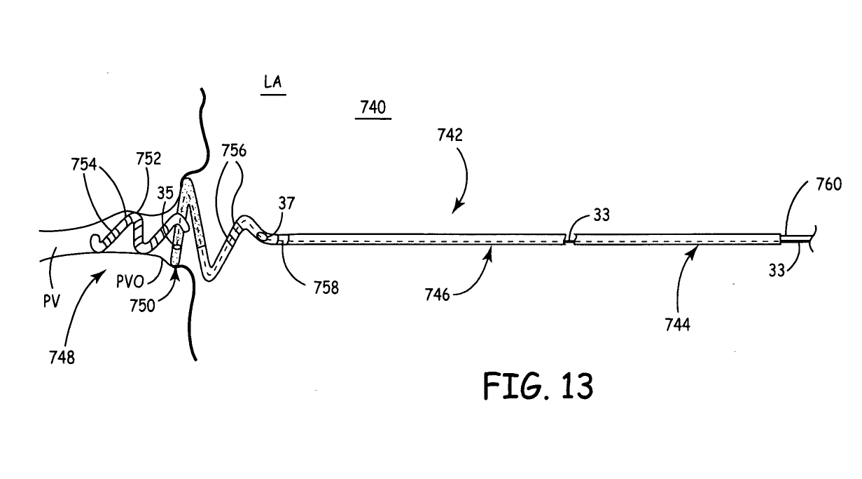

According to the present invention the ablation section 750 thus forms an adjustable-diameter loop catheter apparatus having a loop portion substantially transverse to the longitudinal axis. In the embodiment of FIG. 12, the adjustable-diameter loop formed by the ablation section 750 is greater than a single revolution, such as a curving approximately 360°-540°, but does not necessarily form a decreasing radius helix. The tip 752 extends distally from the ablation section 750 and can form a slightly distally decreasing radius helix. With this configuration, an outer diameter defined by the ablation section 750 is initially greater than an outer dimension of a pulmonary vein ostium (shown in FIG. 13), whereas a maximum outer diameter defined by the tip 752 approximates a diameter of an interior portion of a pulmonary vein (not shown). In use, when tension is applied to the member 33 the diameter of the ablation section 750 is reduced to approximately slightly less than the diameter of the PVO and the tip portion 752 and the ablation section 750 is advanced into the PV. After reaching a desired location within the PV the tension is released or at least reduced so that the ablation section engages the surrounding PV tissue (e.g., PVO or interior PV tissue). Though not illustrated, the tip 752 can form a distal leader, similar to the leader section 726 (FIG. 9) previously described.

As with the catheter assembly 700 (FIG. 9) previously described, the ablation section 750 and the tip 752 define differing shapes. In addition, and in accordance with one embodiment, the ablation section 750 is formed by microporous material as previously described, whereas the tip 752 is fluid impermeable. Thus, the catheter body 742, and in particular the ablation section 750, is configured to ablate tissue by irrigating energized conductive fluid, whereas ablation will not occur along the tip 752. Also, as with the catheter assembly 700 previously described, the catheter body 742 can include port 37 forming the terminus of multi-purpose lumen 29 and thus may be configured to provide the utility of the embodiments previously described. Also, the catheter body 742 can include electrodes 754 positioned along the tip 752; electrodes 756 positioned adjacent, but proximal, the ablation section 750; and an electrode 758 positioned along the intermediate portion 746.

In one embodiment, a shaping wire 760 (shown partially in FIG. 12) is provided to selectively form the distal portion 748 to the shape illustrated in FIG. 12, similar to previously described embodiments. Though not illustrated, one or more coil electrodes are positioned within or along the ablation section 750 at various radial positions. Once again, the coil electrodes energize fluid irrigated through the ablation section 750 during use, and their radial location correlated with radial locations of respective ones of the electrodes 754 along the tip 752. Alternatively, and as previously described, a wide variety of other configurations can be employed to form the distal portion 748 to the shape shown in FIG. 12 and/or to provide ablative energy.

During use, and with reference to FIG. 13, following various preparatory steps, the distal portion 748 is deployed within the LA as previously described. As a point of reference, FIG. 13 depicts the catheter body 740, and in particular the ablation section 750, compressed against the chamber wall W. With this in mind, the tip 752 is first used to locate the PV. Once again, the distally decreasing radius helix form of the tip 752 promotes placement within the PV with minimal trauma to the PV tissue. Once located, the distal portion 748, and in particular, the tip 752 is advanced within the PV until the ablation section 750 contacts the tissue wall W. In this regard, the tip 752 essentially seats within the PV, such that the ablation section 750 is substantially centered about the PVO and seats against the wall W in an extra-ostial position. The catheter 742 of FIG. 13 also can include either or both the elongated diameter adjusting member 33 coupled to the anchor member 35 and/or the multi-purpose lumen (not shown) that terminates at the port 37. Thus, all the previously-described advantages of these components may be practiced in conjunction with the embodiment depicted in FIG. 13.

Once properly positioned, an ablative energy is applied to the tissue wall W via the ablation section 750. Following application of the ablation energy, the electrodes 754-758 are operated to sense electrical activity inside and outside of the pulmonary vein, as previously described. If it is determined that electrical activity continues to traverse the ablated lesion or selected portion(s) of the circumference, an ablation energy can again be applied to further ablate the tissue wall W about the entire PVO or only about selected portions of the pulmonary vein ostium as previously described.

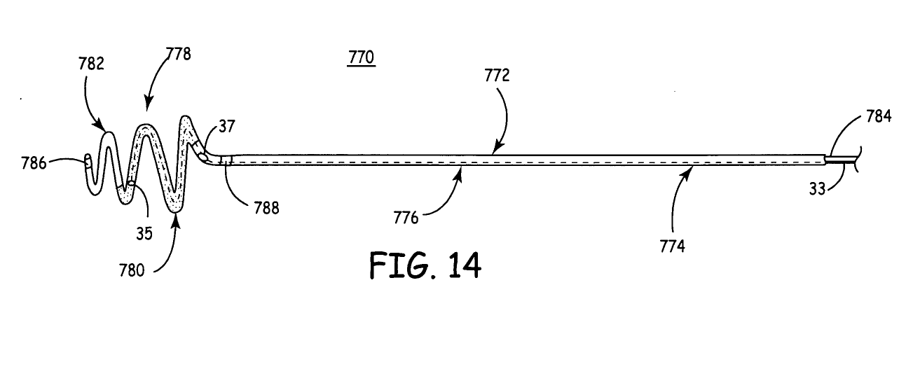

Yet another alternative embodiment catheter assembly 770 is depicted in FIG. 14. For ease of illustration, only a distal region of the catheter assembly 770 is shown. The catheter assembly 770 is similar to the catheter assemblies 700 (FIG. 9) and 740 (FIG. 12) previously described, and includes a catheter body 772 having a proximal portion 774, an intermediate portion 776, and a distal portion 778. The proximal portion 774 is connected to an ablative energy source (not shown). The intermediate portion 776 extends from the proximal portion 774 and defines a longitudinal axis. Finally, the distal portion extends from the intermediate portion 778 and includes an ablation section 780 and a tip 782.

The tip 782 extends distally from the ablation section 780. Further, the ablation section 780 and the tip 782 combine to define a distally decreasing radius helix for the distal portion 778. Thus, unlike the catheter bodies previously described, the ablation section 780 and the tip 782 define a substantially continuously curving shape. However, the ablation section 780 and the tip 782 have other varying features. For example, in one embodiment, the ablation section 780 is formed of a microporous material, such as expanded PTFE, previously described; whereas the tip 782 is formed of a fluid impermeable material. Further, the tip 782 is formed of an atrumatic material such as low durometer elastromer or thermoplastic and/or utilizing a smaller diameter shaping wire 784, and is thus softer than the ablation section 780.

As with previous embodiments, the catheter assembly 770 can incorporate a shaping wire 784 to promote a desired helical or looping shape to the distally decreasing- and adjustable-diameter of the distal section 778. Once again, the shaping wire 784 can carry one or more coil electrodes (not shown) positioned within the ablation section 780. The coil electrodes serve to energize, via an ablative energy source (not shown), fluid irrigated through the ablation section 780. Alternatively, the distally decreasing helical shape of the distal portion 778 can be achieved with something other than the shaping wire 784, for example thermally formed thermoplastics or mechanically manipulated torque and puller wires that create a helical shape. Further, an ablation technique other than energized conductive fluid irrigated through the ablation section 780 can be incorporated into the catheter body 772. Regardless, the catheter body 772 can carry an electrode 786 along the tip 782 and an electrode 788 along the intermediate portion 776. The catheter 742 of FIG. 14 can include either or both the elongated diameter adjusting member 33 coupled to the anchor member 35 (and the corresponding shaping wire—not shown) and/or the multi-purpose lumen (not shown) that terminates at the port 37. Thus, all the previously-described advantages of these components may be practiced in conjunction with the embodiment depicted in FIG. 14.

During use, the distal portion 778 is deployed similar to the embodiments previously described. Once again, the tip 782 is uniquely configured to optimally locate and seat within a pulmonary vein (not shown). This relationship essentially ensures that the ablation section 780, once compressed against the tissue wall is centered about the pulmonary vein ostium (not shown), more particularly, in an extra-ostial location. Finally, the electrodes 786, 788 provide a means for evaluating electrical activity both inside and outside of the pulmonary vein.

The catheter assembly of the present invention provides a highly viable tool for electrically isolating a vessel, such as a pulmonary vein or coronary sinus, from a chamber, such as the left atrium.

With respect to one embodiment in which the distal portion of the catheter body forms a distally decreasing- and adjustable-diameter radius loop, coil or helix, the ablation section is readily and consistently positioned about, promotes physical engagement relative to, and assists removal of an ablation catheter from, a pulmonary vein ostium.

In one aspect of the invention, by forming the distal portion to include both an adjustable-diameter ablation section and a distally extending tip, the pulmonary vein in question is easily located, engaged and disengaged using apparatus according to the present invention. Further, the tip can be formed to seat within the pulmonary vein, thereby providing a user with a tactile confirmation of proper positioning. Finally, reference electrodes can provide both inside and outside of the pulmonary vein to confirm electrical isolation thereof following ablation.

Although the present invention has been described with reference to certain embodiments, workers skilled in the art will recognize that changes may be made in form and detail without departing from the spirit and scope of the invention. For example, the embodiments describe electrical isolation of a pulmonary vein from the left atrium for treatment of atrial fibrillation. Alternatively, the method and apparatus of the present invention may be utilized in the treatment of other cardiac arrhythmias, such as isolating the coronary sinus from the right atrium, the superior vena cava, or isolating the outflow tract (or pulmonary valve) from the right ventricle. Further, certain features can be altered or eliminated while still providing a viable device according to the present invention. For example, the ablation section and tip need not be made of differing materials. Further, a variety of ablative energy sources are available, including ultrasound, RF energy, microwave energy, laser, cryogenic energy, thermal energy, etc. Further, while a shaping wire can be employed, the catheter body itself can be made of a shape memory material able to achieve the desired shape. In addition, the shaping wire may be taper ground to reduce its diameter near the distal end thereof (corresponding to the tip or leader section of the catheter body), thereby reducing the stiffness of the catheter body tip upon final assembly and/or may be entirely contained only in contact with the distal end portion. Even further, the catheter body can be provided with various pull wires, the maneuvering of which selectively forms the distal portion to the desired shape. Finally, other features associated with different embodiments can be incorporated into the catheter assembly hereof. Even further, other features not specifically disclosed can be employed. For example, the catheter assembly may include a rapid exchange feature for quick placement over, and removal from, a guidewire.

While the present invention has been described primarily with reference to diagnostic procedures and therapy provided in and about the PVs of the LA, no such limitation is intended. That is, in addition to the structures herein and the methods of fabrication and use described with respect to and according to certain exemplary embodiments of the present invention other related—albeit sometimes slightly different—structures and the like are intended to be covered by the claims appended hereto. For example, the apparatus of the present invention may be used to map or diagnose electrical activity in a variety of locations and/or ablate tissue.

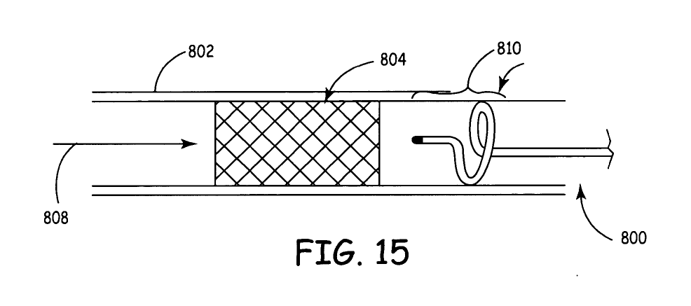

As depicted in FIG. 15, an additional embodiment of the inventive apparatus involves a scaled-down version of the PV mapping and ablation apparatus 800 deployed to ablate interior tissue of a vessel 802 (e.g., a vein) “downstream” (flow depicted by arrow 808) from an implantable medical device such as a stent 804. Following ablation of such tissue diameter of the vessel 802 at the ablated portion 810 of tissue decreases, thereby providing a modicum of support for the stent 804 (or other IMD) so that it is less likely to migrate from its initial intended location in the vessel 802. In this scaled-down form of the present invention the diameter of the catheter prior to deploying the adjustable diameter loop (not depicted) is on the order of less than about two or three French (2-3 F.) as compared to the seven or eight French (7-8 F.) diameter for embodiments of the present invention configures for PV mapping and/or ablation.

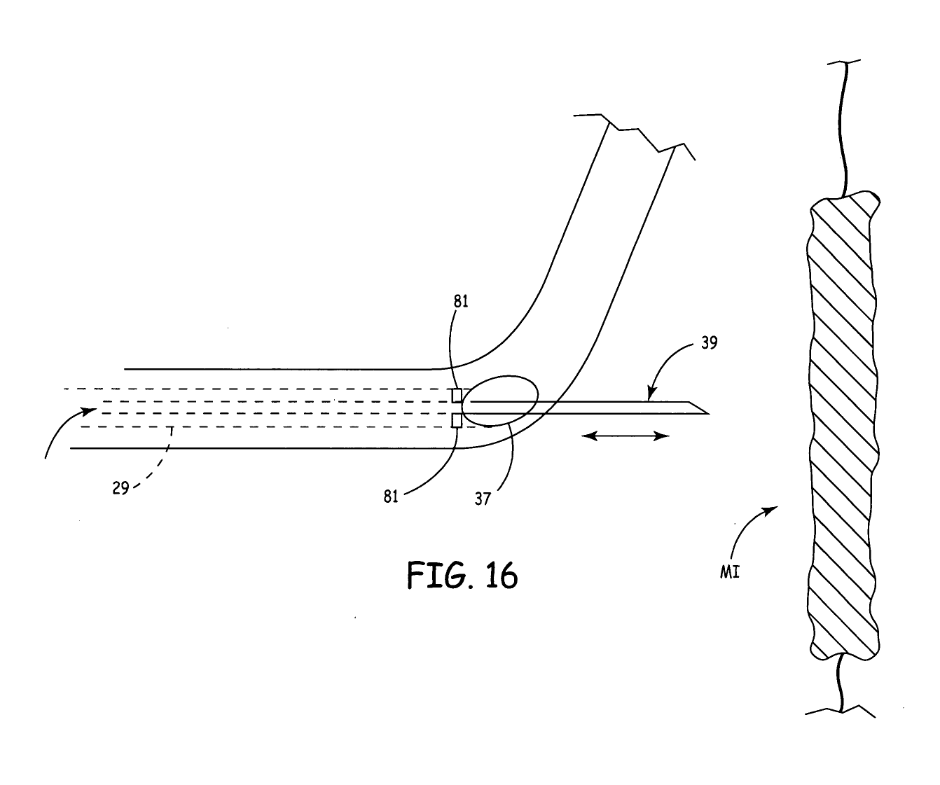

Turning now to FIG. 16, which is an elevational view of a deployable instrument 39 (e.g., a biopsy collection instrument or a fluid dispensing needle) in a partially deployed location from a multi-purpose port 37 forming the terminus of a multi-purpose lumen 29 and disposed next to a volume of tissue (e.g., a myocardial infarct) and illustrating cooperating mechanical stops 81 so that the instrument only deploys a predetermined distance from a port 37. Thus, a sample of tissue from the MI may be harvested for later inspection and testing or a volume of therapeutic material may be injected into said volume of tissue. As with other related embodiments described herein, the instrument 39 can deploy within an area defined by the periphery of a loop portion of the associated catheter (not shown).

Thus a multi-purpose catheter assembly and methods of use have been presented. The scope and breadth of this patent disclosure is to be construed as broadly as reasonably possible as interpreted by one of skill in the art to which this multi-purpose medical device is directed. Said scope and breadth being limited only by the following claims.

1. An elongated catheter assembly having at least one adjustable-diameter loop, comprising:
a distal portion, said distal portion further comprising at least one loop;
means for adjusting a diameter dimension of the at least one loop; and
an electrode section disposed about the at least one loop, said electrode section further comprising a plurality of individually addressable discrete electrically conducting electrode members coupled to at least one elongated electrical conductor adapted for electrical communication with remote circuitry.

2. An elongated catheter assembly according to claim 1, wherein said means for adjusting comprises a one of:
a resilient unitary wire, a braided wire cable, a coiled wire cable, and wherein said means for adjusting is slideably disposed within said distal portion.

3. An elongated catheter assembly according to claim 2, further comprising a sheath slideably coupled around said means for adjusting.

4. An elongated catheter assembly according to claim 1, further comprising at least one lumen formed through at least the distal portion.

5. An elongated catheter assembly according to claim 4, further comprising a fluid reservoir removeably coupled to a proximal end portion of the irrigation/guide-wire lumen.

6. An elongated catheter assembly according to claim 4, further comprising a contrast media lumen.

7. An elongated catheter assembly according to claim 4, further comprising a fluid reservoir removeably coupled to the at least one lumen.

8. An elongated catheter assembly according to claim 2, wherein said means for adjusting is slideably coupled within an inner circumference of an interior wall of the second conduit.

9. An elongated catheter assembly according to claim 1, further comprising a tip portion extending distally from the at least one loop.

10. An elongated catheter assembly according to claim 1, further comprising a remote electronic signal processing unit adapted to be electrically coupled to the plurality of individually addressable discrete electrically conducting electrode members.

11. An elongated catheter assembly according to claim 10, wherein:
upon activation of a tissue mapping circuit the remote electronic signal processing unit measures electrical signals from the electrode members; and
upon activation of a tissue ablation circuit the remote electronic signal processing unit provides a predetermined amount of ablation energy to form a tissue lesion region proximate one or more of the electrode members.

12. An elongated catheter assembly according to claim 1, wherein said at least one loop comprises a helical loop member and wherein each successive one of the series of helical loop members has a smaller diameter dimension than a prior helical loop member.

13. An elongated catheter apparatus, comprising:
an elongated catheter body;
a helical distal portion coupled to the elongated catheter body;
an elongated diameter-adjusting member disposed within the elongated catheter body and the helical distal portion and having a distal end mechanically coupled proximal to a distal end portion of the helical distal portion so that a diameter dimension of the helical distal portion decreases when tension is applied to the elongated diameter-adjusting member;
at least one electrode mechanically coupled to a portion of the helical distal portion; and
a multi-purpose lumen disposed within said catheter body from a proximal end to a location just proximal the helical distal portion and wherein a longitudinal axis of said lumen is disposed substantially orthogonal to the plane defined by the helical distal portion.

14. An elongated catheter apparatus according to claim 13, further comprising a shaping wire mechanically coupled to at least the helical distal portion.

15. An elongated catheter apparatus according to claim 14, wherein said shaping wire comprises a superelastic material.

16. An elongated catheter apparatus according to claim 14, wherein said shape memory material comprises a nitinol material.

17. An elongated catheter apparatus according to claim 14, wherein said shaping wire is mechanically anchored to a distal tip of the elongated catheter apparatus.

18. An elongated catheter apparatus according to claim 17, wherein said shaping wire slideably couples to an inner circumference of the helical distal portion.

19. An elongated catheter apparatus according to claim 13, wherein said helical distal portion comprises a successively increasing diameter helix.

20. An elongated catheter apparatus 13, further comprising a pull wire coupled to a location proximal the helical distal portion so that when tension is applied to said pull wire the helical distal portion deflects from a longitudinal axis of said intermediate portion.

21. An elongated catheter apparatus according to claim 13, further comprising a plurality of electrodes mechanically coupled to the helical distal portion and electrically coupled to the at least one conductor.

22. An elongated catheter apparatus according to claim 13, further comprising at least a one of:
a contrast media fluid source fluidly coupled to said multi-purpose lumen;
a manually deployable biopsy instrument disposed in said multi-purpose lumen; and
a manually deployable fluid injecting instrument disposed in said multi-purpose lumen.

23. An elongated catheter apparatus according to claim 13, further comprising an irrigation fluid conduit formed through at least the elongated catheter body.

24. An elongated catheter apparatus according to claim 23, further comprising a fluid reservoir removeably coupled to a proximal end of either the multi-purpose lumen or the irrigation fluid conduit.

25. An elongated catheter apparatus according to claim 24, further comprising a diagnostic electrical signal processor or an ablation signal generator coupled to the second end of the at least one conductor.

26. An elongated catheter assembly according to claim 1, wherein at least a segment of the distal portion comprises a distally decreasing radius helix.

27. An elongated catheter apparatus according to claim 23, wherein a distal end portion of the irrigation lumen terminates distal the at least two electrodes and wherein at least a portion of the distal end portion of the irrigation lumen is formed of a microporous polymer material.

28. A catheter assembly of claim 27, wherein the microporous polymer material comprises a high-density, expanded PTFE material.

29. A catheter assembly of claim 13, wherein the at least two electrodes comprise a coil electrode.

30. A method of reaching a desired target through a confined vessel with a deployable electrical catheter having electrodes disposed on an adjustable-diameter loop portion and remotely adjusting the diameter of said loop portion, comprising:
advancing a delivery catheter containing a deployable electrical catheter assembly through a confined vessel to a location adjacent a desired target, said deployable electrical catheter assembly including a proximal portion, an intermediate portion, and a temporarily collapsed, expandable distal portion;
withdrawing the delivery catheter until at least the temporarily collapsed, expandable distal portion extends from the distal end of said delivery catheter, said temporarily collapsed, expandable distal portion further comprising:
an adjustable-diameter loop portion that in an expanded state is oriented substantially transverse to the longitudinal axis of the intermediate portion;
means for expanding the adjustable-diameter loop portion from a compact collapsed state to the expanded state;
a loop diameter-adjustment assembly slideably coupled to the proximal portion, the intermediate portion and the distal portion, and wherein a first end of the loop diameter-adjustment assembly connects to a distal part of the adjustable-diameter loop portion and a manually accessible second end; and
at least one electrode coupled to the adjustable-diameter loop portion;
expanding the loop portion to a predetermined curvilinear shape by operation of the means for expanding;
manipulating the loop diameter-adjustment assembly to adjust a diameter dimension of the loop portion to a desired dimension;
advancing the loop portion such that at least a portion of the adjustable-diameter loop portion contacts the desired target; and
activating a remote signal processor or signal generator to respectively diagnose an electrical condition of, or ablate a lesion pattern on, the desired target.

31. A method according to claim 30, wherein the means for expanding the adjustable-diameter loop portion from a compact collapsed state to the expanded state comprises at least a one of:
an elongated shape memory material disposed within at least the adjustable-diameter loop portion,
a wire connected to the distal part of the adjustable-diameter loop portion,
a resilient elongated member connected to the distal part of the adjustable-diameter loop portion.

32. A method according to claim 30, wherein the loop diameter-adjustment assembly further comprises a one of:
a resilient unitary wire,
a braided wire cable,
a coiled wire cable, and
wherein said loop diameter-adjustment assembly slideably couples to the proximal portion, the intermediate portion and the distal portion.

33. An adjustable-diameter catheter assembly, comprising:
a distal portion having at least one longitudinal conduit and at least one arcuate portion, said arcuate portion including a first relative diameter;
a multi-purpose lumen disposed from a proximal portion to a location proximal said distal portion; and
an elongated diameter-adjusting member disposed in the at least one longitudinal conduit and mechanically coupled to said distal portion proximal an end portion of the arcuate portion,
wherein tension applied to the elongated diameter-adjusting member causes a decrease in the first relative diameter of the arcuate portion.

34. A multi-purpose catheter, comprising:
a catheter body;
a multi-purpose lumen disposed in said catheter body through an intermediate portion thereof;
a port fluidly coupling the multi-purpose lumen disposed at the terminus of the intermediate portion;
a distal portion coupled to the end of the intermediate portion;
wherein said distal portion further comprises:
at least one loop portion; and
at least two electrodes are coupled to said at least one loop portion.

35. A multi-purpose catheter according to claim 34, wherein said multi-purpose lumen is adapted to at least temporarily receive each of the following:
an elongated guide-wire;
an elongated tissue collection instrument;
an elongated tissue-piercing, fluid dispensing instrument;
a volume of contrast media;
a volume of a fluid material.

36. A multi-purpose catheter according to claim 34, wherein said port further comprises a valve member disposed over all or substantially the entire open surface area of said port.

37. A multi-purpose catheter according to claim 36, wherein said valve member comprises at least two substantially opposing flaps.

38. A multi-purpose catheter according to claim 34, wherein the distal portion of the catheter has a reduced diameter relative to an intermediate or proximal portion of the catheter.

39. A multi-purpose catheter according to claim 34, wherein said catheter is sized for access of a vessel of a body to ablate tissue either adjacent at least one stent previously deployed in said vessel or at a location wherein a stent is scheduled to be deployed.

40. A multi-purpose catheter according to claim 35, wherein said catheter is disposed adjacent a myocardiac infarction and either the elongated biopsy collection instrument is deployable therefrom or the elongated tissue-piercing, fluid dispensing instrument is deployable therefrom.
